(19) United States
(12) Patent Application Publication
(10) Pub. No.: US 2015/0283236 A1
(43) Pub. Date: Oct. 8, 2015

(54) METHODS FOR TREATING SUBJECTS WITH PRIMARY HYPERCHOLESTEROLEMIA THAT IS NOT ADEQUATELY CONTROLLED

(71) Applicants: Marie Baccara-Dinet, Paris (FR); Corinne Hanotin, Paris (FR); Yuko Harada, Tokyo (JP); Toshiaki Sato, Tokyo (JP); Hideyo Suzuki, Tokyo (JP)

(72) Inventors: Marie Baccara-Dinet, Paris (FR); Corinne Hanotin, Paris (FR); Yuko Harada, Tokyo (JP); Toshiaki Sato, Tokyo (JP); Hideyo Suzuki, Tokyo (JP)

(21) Appl. No.: 14/657,186

(22) Filed: Mar. 13, 2015

Related U.S. Application Data
(60) Provisional application No. 62/080,709, filed on Nov. 17, 2014.
(60) Provisional application No. 61/954,107, filed on Mar. 17, 2014.

(30) Foreign Application Priority Data
Feb. 26, 2015 (EP) 15305292.3

(51) Int. Cl. A61K 39/395; A61K 45/06

(52) U.S. Cl. CPC A61K 39/3955; A61K 45/06; A61K 2039/505

(57) ABSTRACT

The present invention provides methods for treating hypercholesterolemia. The methods of the present invention comprise administering to a patient a pharmaceutical composition comprising a PCSK9 inhibitor. In certain embodiments, the PCSK9 inhibitor is an anti-PCSK9 antibody such as the exemplary antibody referred to herein as mAb316P. The methods of the present invention are useful for treating patients with hypercholesterolemia that is not adequately controlled by moderate-dose statin therapy.

### RELATED APPLICATIONS

This application claims the benefit of U.S. Provisional Application No. 62/080,709, filed on Nov. 17, 2014, U.S. Provisional Application No. 61/954,107, filed on Mar. 17, 2014 and European Patent Application No. 15305292.3, filed on Feb. 26, 2015, the contents of which are incorporated herein by reference in their entirety.

### FIELD OF THE INVENTION

The present invention relates to the field of therapeutic treatments of diseases and disorders that are associated with elevated levels of lipids and lipoproteins. More specifically, the invention relates to the use of PCSK9 inhibitors to treat patients with primary hypercholesterolemia that is not adequately controlled by a lipid-modifying therapy, including a moderate-dose statin therapy.

### BACKGROUND

Hypercholesterolemia, particularly an increase in low-density lipoprotein (LDL) cholesterol (LDL-C) levels, constitutes a major risk for the development of atherosclerosis and coronary heart disease (CHD) (Sharrett et al., 2001, Circulation 104:1108-1113). Low-density lipoprotein cholesterol is identified as the primary target of cholesterol lowering therapy and is accepted as a valid surrogate therapeutic endpoint. Numerous studies have demonstrated that reducing LDL-C levels reduces the risk of CHD with a strong direct relationship between LDL-C levels and CHD events; for each 1 mmol/L (˜40 mg/dL) reduction in LDL-C, cardiovascular disease (CVD) mortality and morbidity is lowered by 22%. Greater reductions in LDL-C produce greater reduction in events, and comparative data of intensive versus standard statin treatment suggest that the lower the LDL-C level, the greater the benefit in patients at very high cardiovascular (CV) risk.

Current LDL-C lowering medications include statins, cholesterol absorption inhibitors (e.g., ezetimibe [EZE]), fibrates, niacin, and bile acid sequestrants. Statins are the most commonly prescribed, as they have shown a greater ability to lower LDL-C and reduce CHD events. However, many patients at risk of cardiovascular disease (CVD) have poorly controlled low-density lipoprotein cholesterol (LDL-C) despite statin therapy.

### BRIEF SUMMARY OF THE INVENTION

The present invention provides methods for treating hypercholesterolemia. In particular, the methods of the present invention are useful for treating subjects with primary hypercholesterolemia that is not adequately controlled by a background lipid-modifying therapy, including a moderate-dose statin therapy.

According to one aspect, the methods of the present invention comprise administering one or more doses of a PCSK9 inhibitor to a subject with primary hypercholesterolemia and LDL-C ≧100 mg/dL that is not adequately controlled by a background lipid-modifying therapy, including a moderate-dose statin therapy (i.e., hypercholesterolemia that is not adequately controlled by moderate-dose statin therapy in the absence of a PCSK9 inhibitor, with or without other lipid modifying therapy). According to certain embodiments of the present invention, the PCSK9 inhibitor is administered to the patient as an add-on therapy to the patient's existing statin therapy.

According to another aspect, the methods of the present invention comprise selecting a patient who is on a therapeutic regimen comprising a daily dose of a statin (e.g., a moderate-dose statin therapy), and administering to the patient one or more doses of a PCSK9 inhibitor in combination with (i.e., “on top of”) the statin therapy.

The present invention also provides pharmaceutical compositions comprising a PCSK9 inhibitor for use in treating a patient with primary hypercholesterolemia and LDL-C ≧100 mg/dL that is not controlled by moderate-dose statin therapy and a pharmaceutically acceptable carrier.

An aspect of the invention includes a method for treating hypercholesterolemia in a subject in need thereof, comprising: a) selecting a subject who is currently being treated for hypercholesterolemia by administration of a background lipid-modifying therapy; b) continuing treating the subject with the background lipid-modifying therapy; and c) administering to the subject a pharmaceutical composition comprising about 75 mg of an antibody or antigen binding fragment thereof that specifically binds PCSK9 at a frequency of about once every other week (q2w), thereby treating the hypercholesterolemia in the subject.

According to one aspect, the method lowers the subject's low density lipoprotein-C (LDL-C) level by at least 50%. According to another aspect, the method lowers the subject's low density lipoprotein-C (LDL-C) level by at least 55%. According to another aspect, the method lowers the subject's low density lipoprotein-C (LDL-C) level by at least 60%. According to another aspect, the method lowers the subject's low density lipoprotein-C (LDL-C) level by about 62%.

According to one aspect, the subject, prior to or at the time of administration of the pharmaceutical composition, exhibits hypercholesterolemia defined as a serum low-density lipoprotein cholesterol (LDL-C) level of greater than about 70 mg/dL. According to another aspect, the subject, prior to or at the time of administration of the pharmaceutical composition, exhibits hypercholesterolemia defined as a serum low-density lipoprotein cholesterol (LDL-C) level of greater than about 100 mg/dL.

According to one aspect, the subject has heterozygous Familial Hypercholesterolemia (heFH). According to another aspect, the subject has a form of hypercholesterolemia that is not Familial Hypercholesterolemia (nonFH).

According to one aspect, the background lipid-modifying therapy comprises a therapeutic agent selected from the group consisting of a statin, ezetimibe, a fibrate, niacin, an omega-3 fatty acid, and a bile acid resin. According to one aspect, the statin is selected from the group consisting of cerivastatin, atorvastatin, simvastatin, pitavastatin, rosuvastatin, fluvastatin, lovastatin and pravastatin.

According to one aspect, the pharmaceutical composition is administered for at least 24 weeks.

According to one aspect, the pharmaceutical composition is administered subcutaneously.

According to one aspect, the antibody or antigen binding fragment thereof comprises the heavy and light chain CDRs of a HCVR/LCVR amino acid sequence pair selected from the group consisting of SEQ ID NOs: 1/6 and 11/15. According to another aspect, the antibody or antigen-binding fragment thereof comprises heavy and light chain CDR amino acid sequences having SEQ ID NOs: 12, 13, 14, 16, 17, and 18. According to an additional aspect, the antibody or antigen-binding fragment thereof comprises an HCVR having the amino acid sequence of SEQ ID NO: 11 and an LCVR having the amino acid sequence of SEQ ID NO: 15. According to a further aspect, the antibody or antigen-binding fragment thereof comprises heavy and light chain CDR amino acid sequences having SEQ ID NOs: 2, 3, 4, 7, 8, and 10. According to yet a further aspect, the antibody or antigen-binding fragment thereof comprises an HCVR having the amino acid sequence of SEQ ID NO: 1 and an LCVR having the amino acid sequence of SEQ ID NO: 6. According to yet a further aspect, the antibody or antigen-binding fragment thereof binds to the same epitope on PCSK9 as an antibody comprising heavy and light chain CDR amino acid sequences having SEQ ID NOs: 12, 13, 14, 16, 17, and 18; or SEQ ID NOs: 2, 3, 4, 7, 8, and 10. According to an alternative aspect, the antibody or antigen-binding fragment thereof competes for binding to PCSK9 with an antibody comprising heavy and light chain CDR amino acid sequences having SEQ ID NOs: 12, 13, 14, 16, 17, and 18; or SEQ ID NOs: 2, 3, 4, 7, 8, and 10.

An aspect of the invention includes a method for reducing low density lipoprotein-C (LDL-C) in a subject in need thereof, comprising: a) selecting a subject who is currently being treated for hypercholesterolemia by administration of a background lipid-modifying therapy; b) continuing treating the subject with the background lipid-modifying therapy; and c) administering to the subject a pharmaceutical composition comprising about 75 mg of an antibody or antigen binding fragment thereof that specifically binds PCSK9 at a frequency of about once every other week (q2w), thereby reducing the LDL-C in the subject.

According to one aspect, the method lowers the subject's low density lipoprotein-C (LDL-C) level by at least 50%. According to another aspect, the method lowers the subject's low density lipoprotein-C (LDL-C) level by at least 60%.

According to one aspect, the subject, prior to or at the time of administration of the pharmaceutical composition, exhibits hypercholesterolemia defined as a serum low-density lipoprotein cholesterol (LDL-C) level of greater than about 70 mg/dL. According to another aspect, the subject, prior to or at the time of administration of the pharmaceutical composition, exhibits hypercholesterolemia defined as a serum low-density lipoprotein cholesterol (LDL-C) level of greater than about 100 mg/dL.

According to one aspect, the subject has heterozygous Familial Hypercholesterolemia (heFH). According to another aspect, the subject has a form of hypercholesterolemia that is not Familial Hypercholesterolemia (nonFH).

According to one aspect, the background lipid-modifying therapy comprises a therapeutic agent selected from the group consisting of a statin, ezetimibe, a fibrate, niacin, an omega-3 fatty acid, and a bile acid resin. According to one aspect, the statin is selected from the group consisting of cerivastatin, atorvastatin, simvastatin, pitavastatin, rosuvastatin, fluvastatin, lovastatin and pravastatin.

According to one aspect, the pharmaceutical composition is administered for at least 24 weeks.

According to one aspect, the pharmaceutical composition is administered subcutaneously.

According to one aspect, the antibody or antigen binding fragment thereof comprises the heavy and light chain CDRs of a HCVR/LCVR amino acid sequence pair selected from the group consisting of SEQ ID NOs: 1/6 and 11/15. According to another aspect, the antibody or antigen-binding fragment thereof comprises heavy and light chain CDR amino acid sequences having SEQ ID NOs: 12, 13, 14, 16, 17, and 18. According to an additional aspect, the antibody or antigen-binding fragment thereof comprises an HCVR having the amino acid sequence of SEQ ID NO: 11 and an LCVR having the amino acid sequence of SEQ ID NO: 15. According to a further aspect, the antibody or antigen-binding fragment thereof comprises heavy and light chain CDR amino acid sequences having SEQ ID NOs: 2, 3, 4, 7, 8, and 10. According to yet a further aspect, the antibody or antigen-binding fragment thereof comprises an HCVR having the amino acid sequence of SEQ ID NO: 1 and an LCVR having the amino acid sequence of SEQ ID NO: 6. According to yet a further aspect, the antibody or antigen-binding fragment thereof binds to the same epitope on PCSK9 as an antibody comprising heavy and light chain CDR amino acid sequences having SEQ ID NOs: 12, 13, 14, 16, 17, and 18; or SEQ ID NOs: 2, 3, 4, 7, 8, and 10. According to an alternative aspect, the antibody or antigen-binding fragment thereof competes for binding to PCSK9 with an antibody comprising heavy and light chain CDR amino acid sequences having SEQ ID NOs: 12, 13, 14, 16, 17, and 18; or SEQ ID NOs: 2, 3, 4, 7, 8, and 10.

An aspect of the invention includes a method for reducing low density lipoprotein-C (LDL-C) by at least 50% in a subject in need thereof, comprising: a) selecting a subject who is currently being treated for hypercholesterolemia by administration of a background lipid-modifying therapy; b) continuing treating the subject with the background lipid-modifying therapy; and c) administering to the subject a pharmaceutical composition comprising about 75 mg of an antibody or antigen binding fragment thereof that specifically binds PCSK9 at a frequency of about once every other week (q2w), thereby reducing the LDL-C in the subject by at least 50%.

An aspect of the invention includes a method for improving one or more hypercholesterolemia-associated parameters in a subject in need thereof comprising administering to the subject a pharmaceutical composition comprising about 75 mg of an antibody or antigen binding fragment thereof that specifically binds PCSK9 at a frequency of about once every other week (q2w), wherein the subject is treated concurrently with a background lipid-modifying therapy, and wherein the improvement in a hypercholesterolemia-associated parameter is selected from the group consisting of: (a) a decrease from baseline of low density lipoprotein-C (LDL-C); (b) a decrease from baseline of total cholesterol (TC); and (c) a decrease from baseline of lipoprotein (a).

According to one aspect, the improvement in a hypercholesterolemia-associated parameter is a decrease from baseline of LDL-C of at least 50%. According to another aspect, the improvement in a hypercholesterolemia-associated parameter is a decrease from baseline of LDL-C of at least 55%. According to another aspect, the improvement in a hypercholesterolemia-associated parameter is a decrease from baseline of LDL-C of at least 60%. According to another aspect, the improvement in a hypercholesterolemia-associated parameter is a decrease from baseline of LDL-C of about 62%. According to an additional aspect, the improvement in a hypercholesterolemia-associated parameter is a decrease from baseline of TC of at least 30%. According to a further aspect, the improvement in a hypercholesterolemia-associated parameter is a decrease from baseline of TC of at least 35%. According to another aspect, the improvement in a hypercholesterolemia-associated parameter is a decrease from baseline of TC of about 36%.

According to one aspect, the improvement in a hypercholesterolemia-associated parameter is a decrease from baseline of lipoprotein (a) of at least 30%. According to another aspect, the improvement in a hypercholesterolemia-associated parameter is a decrease from baseline of lipoprotein (a) of at least 35%. According to another aspect, the improvement in a hypercholesterolemia-associated parameter is a decrease from baseline of lipoprotein (a) of at least 40%. According to another aspect, the improvement in a hypercholesterolemia-associated parameter is a decrease from baseline of lipoprotein (a) of about 42%.

An aspect of the invention provides a method for lowering serum low-density lipoprotein cholesterol (LDL-C) in a subject in need thereof, comprising: a) selecting a patient with hypercholesterolemia who is currently being treated for hypercholesterolemia by administration of a background moderate-dose statin therapy, and who is not adequately controlled by moderate-dose statin therapy; b) continuing to treat the subject with the moderate-dose statin therapy; and c) administering to the subject a pharmaceutical composition comprising about 75 mg of an antibody or antigen binding fragment thereof that specifically binds PCSK9 at a frequency of about once every other week (q2w), thereby lowering the serum LDL-C level by at least 50%.

According to one aspect, the subject exhibits a serum LDL-C level of greater than about 70 mg/dL. According to another aspect, the subject exhibits a serum LDL-C level of greater than about 100 mg/dL. According to another aspect, the subject's serum LDL-C level is lowered by at least 60%. According to another aspect, the subject has heterozygous Familial Hypercholesterolemia (heFH). According to another aspect, the subject has a form of hypercholesterolemia that is not Familial Hypercholesterolemia (nonFH).

According to one aspect, the subject is also being treated with a non-statin background lipid-modifying therapy comprising a therapeutic agent selected from the group consisting of ezetimibe, a fibrate, niacin, an omega-3 fatty acid, and a bile acid resin. According to another aspect, the statin is selected from the group consisting of cerivastatin, atorvastatin, simvastatin, pitavastatin, rosuvastatin, fluvastatin, lovastatin and pravastatin. According to another aspect, the pharmaceutical composition is administered for at least 24 weeks. According to another aspect, the pharmaceutical composition is administered subcutaneously.

According to one aspect, the antibody or antigen binding fragment thereof comprises the heavy and light chain CDRs of a HCVR/LCVR amino acid sequence pair selected from the group consisting of SEQ ID NOs: 1/6 and 11/15. According to another aspect, the antibody or antigen-binding fragment thereof comprises heavy and light chain CDR amino acid sequences having SEQ ID NOs: 12, 13, 14, 16, 17, and 18. According to another aspect, the antibody or antigen-binding fragment thereof comprises an HCVR having the amino acid sequence of SEQ ID NO:11 and an LCVR having the amino acid sequence of SEQ ID NO:15. According to another aspect, the antibody or antigen-binding fragment thereof comprises heavy and light chain CDR amino acid sequences having SEQ ID NOs: 2, 3, 4, 7, 8, and 10. According to another aspect, the antibody or antigen-binding fragment thereof comprises an HCVR having the amino acid sequence of SEQ ID NO:1 and an LCVR having the amino acid sequence of SEQ ID NO:6. According to another aspect, the antibody or antigen-binding fragment thereof binds to the same epitope on PCSK9 as an antibody comprising heavy and light chain CDR amino acid sequences having SEQ ID NOs: 12, 13, 14, 16, 17, and 18; or SEQ ID NOs: 2, 3, 4, 7, 8, and 10. According to another aspect, the antibody or antigen-binding fragment thereof competes for binding to PCSK9 with an antibody comprising heavy and light chain CDR amino acid sequences having SEQ ID NOs: 12, 13, 14, 16, 17, and 18; or SEQ ID NOs: 2, 3, 4, 7, 8, and 10.

Other embodiments of the present invention will become apparent from a review of the ensuing detailed description.

### BRIEF DESCRIPTION OF THE FIGURES

FIG. 1A is a graphic representation of the study design for Option 1—patients receiving atorvastatin 5-20 mg at stable dose for at least 6 weeks prior to screening. FIG. 1B is a graphic representation of the study design for Option 2—patients receiving a lipid lowering treatment other than atorvastatin, or not at stable dose of atorvastatin 5-20 mg for 6 weeks prior to screening.

FIG. 2 is a graph showing LDL-C mean (±SE) percent change from baseline versus time for 12 weeks of treatment period and 8 weeks of follow-up period.

FIG. 3 shows graphs of the proportion of patients achieving: (A) LDL-C<100 mg/dL at Week 12; and (B)≧50% reduction in LDL-C at Week 12 of the clinical trial disclosed herein. n=25 in all groups.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

### Hypercholesterolemia not Adequately Controlled

The present invention relates generally to methods and compositions for treating patients who have hypercholesterolemia that is not adequately controlled by a lipid-modifying therapy. In certain embodiments, the patient is on a daily dose of a statin, such as a moderate-dose of a statin. As used herein, the expression “primary hypercholesterolemia” means that the patient has a plasma cholesterol level in excess of 250 mg/dL prior to taking any medication to treat the disease. As used herein, the expression “not adequately controlled,” in reference to hypercholesterolemia, means that the patient's serum low-density lipoprotein cholesterol (LDL-C) concentration, total cholesterol concentration, and/or triglyceride concentration is not reduced to a recognized, medically-acceptable level (taking into account the patient's relative risk of coronary heart disease) after at least 4 weeks on a therapeutic regimen of a lipid-modifying therapy. For example, “a patient with hypercholesterolemia that is not adequately controlled by a statin” includes patients with a serum LDL-C concentration of greater than about 70 mg/dL, 100 mg/dL, 130 mg/dL, 140 mg/dL, or more (depending on the patient's underlying risk of heart disease) after the patient has been on a stable daily lipid-modifying therapy for at least 4 weeks.

According to certain embodiments, the patient who is treatable by the methods of the present invention has primary hypercholesterolemia (e.g., a serum LDL-C concentration greater than or equal to 100 mg/dL) despite taking a lipid-modifying therapy. In some embodiments, the patient is taking a daily dose of a statin (with or without other lipid-modifying therapy) for at least 4 weeks, 5 weeks, 6 weeks, or more. In certain embodiments, the patient's primary hypercholesterolemia is inadequately controlled by a moderate-dose statin therapy (also referred to herein as “a daily moderate-dose therapeutic statin regimen”).

As used herein, “moderate-dose statin therapy” or “daily moderate-dose therapeutic statin regimen,” means a therapeutic regimen comprising the administration of daily dose of a statin that is below the maximally tolerated dose for a particular patient. (Maximally tolerated dose means the highest dose of statin that can be administered to a patient without causing unacceptable adverse side effects in the patient). A moderate dose of a statin may also be referred to herein as a “submaximal dose.” “Moderate-dose statin therapy” includes but is not limited to, e.g., 5 mg of atorvastatin daily, 10 mg of atorvastatin daily, 15 mg of atorvastatin daily, and 20 mg of atorvastatin daily.

The present invention also includes methods for treating patients with primary hypercholesterolemia that is not adequately controlled by moderate-dose statin therapy comprising daily administration of other statins such as rosuvastatin, cerivastatin, pitavastatin, fluvastatin, lovastatin, and pravastatin.

### Patient Selection

The present invention includes methods and composition useful for treating patients who have primary hypercholesterolemia that is not adequately controlled by a background lipid-modifying therapy, including a daily moderate-dose therapeutic statin regimen. The patients who are treatable by the methods of the present invention may also exhibit one or more of additional selection criteria. For example, a patient may be selected for treatment with the methods of the present invention if the patient is diagnosed with or identified as being at risk of developing a hypercholesterolemia condition such as, e.g., heterozygous Familial Hypercholesterolemia (heFH), homozygous Familial Hypercholesterolemia (hoFH), Autosomal Dominant Hypercholesterolemia (ADH, e.g., ADH associated with one or more gain-of-function mutations in the PCSK9 gene), as well as incidences of hypercholesterolemia that are distinct from Familial Hypercholesterolemia (nonFH).

According to certain embodiments, the patient may be selected on the basis of having a history of coronary heart disease (CHD). As used herein a “history of CHD” (or “documented history of CHD”) includes one or more of: (i) acute myocardial infarction (MI); (ii) silent MI; (iii) unstable angina; (iv) coronary revascularization procedure (e.g., percutaneous coronary intervention [PCI] or coronary artery bypass graft surgery [CABG]); and/or (v) clinically significant CHD diagnosed by invasive or non-invasive testing (such as coronary angiography, stress test using treadmill, stress echocardiography or nuclear imaging).

According to certain embodiments, the patient may be selected on the basis of having non-coronary heart disease cardiovascular disease (“non-CHD CVD”). As used herein, “non-CHD CVD” includes one or more of: (i) documented previous ischemic stroke with a focal ischemic neurological deficit that persisted more than 24 hours, considered as being of atherothrombotic origin; (ii) peripheral arterial disease; (iii) abdominal aortic aneurysm; (iv) atherosclerotic renal artery stenosis; and/or (v) carotid artery disease (transient ischemic attacks or >50% obstruction of a carotid artery).

According to certain embodiments, the patient may be selected on the basis of having one or more additional risk factors such as, e.g., (i) documented moderate chronic kidney disease (CKD) as defined by 30≦seGFR<60 mL/min/1.73 m2 for 3 months or more; (ii) type 1 or type 2 diabetes mellitus with or without target organ damage (e.g., retinopathy, nephropathy, microalbuminuria); (iii) a calculated 10-year fatal CVD risk SCORE≧5% (ESC/EAS Guidelines for the management of dyslipidemias, Conroy et al., 2003, Eur. Heart J. 24:987-1003).

According to certain embodiments, the patient may be selected on the basis of having one or more additional risk factors selected from the group consisting of age (e.g., older than 40, 45, 50, 55, 60, 65, 70, 75, or 80 years), race, national origin, gender (male or female), exercise habits (e.g., regular exerciser, non-exerciser), other preexisting medical conditions (e.g., type-II diabetes, high blood pressure, etc.), and current medication status (e.g., currently taking beta blockers, niacin, ezetimibe, fibrates, omega-3 fatty acids, bile acid resins, etc.).

According to certain embodiments, patients may be selected on the basis of a combination of one or more of the foregoing selection criteria or therapeutic characteristics. For example, according to certain embodiments, a patient suitable for treatment with the methods of the present invention, in addition to having hypercholesterolemia that is not adequately controlled by a daily moderate-dose therapeutic statin regimen, may further be selected on the basis of having heFH or non-FH in combination with: (i) a history of documented CHD, (ii) non-CHD CVD, and/or (iii) diabetes mellitus with target organ damage; such patients may also be selected on the basis of having a serum LDL-C concentration of greater than or equal to 100 mg/dL.

According to certain other embodiments, a patient suitable for treatment with the methods of the present invention, in addition to having hypercholesterolemia that is not adequately controlled by a background lipid-modifying therapy, including a daily moderate-dose therapeutic statin regimen, may further be selected on the basis of having heFH or non-FH without CHD, or non-CHD CVD, but having either (i) a calculated 10-year fatal CVD risk SCORE≧5%; or (ii) diabetes mellitus without target organ damage; such patients may also be selected on the basis of having a serum LDL-C concentration of greater than or equal to 100 mg/dL.

### Administration of a PCSK9 Inhibitor as Add-On Therapy to Moderate-Dose Statin Therapy

The present invention includes methods wherein a patient with hypercholesterolemia that is not adequately controlled by a stable lipid-modifying therapy in the absence of a PCSK9 inhibitor is administered a PCSK9 inhibitor according to a particular dosing amount and frequency, and wherein the PCSK9 inhibitor is administered as an add-on to the patient's therapeutic lipid-modifying regimen. For example, according to certain embodiments, if a patient has primary hypercholesterolemia that is not adequately controlled despite being on a stable daily moderate-dose therapeutic statin regimen comprising, e.g., 20 mg of atorvastatin, the patient may be administered a PCSK9 inhibitor at a particular amount and dosing interval while the patient continues his or her stable daily therapeutic statin regimen (e.g., 20 mg of atorvastatin daily).

The methods of the present invention include add-on therapeutic regimens wherein the PCSK9 inhibitor is administered as add-on therapy to the same stable daily moderate-dose therapeutic statin regimen (i.e., same dosing amount of statin) that the patient was on prior to receiving the PCSK9 inhibitor. In other embodiments, the PCSK9 inhibitor is administered as add-on therapy to a daily moderate-dose therapeutic statin regimen comprising a statin in an amount that is more than or less than the dose of stain the patient was on prior to receiving the PCSK9 inhibitor. For example, after starting a therapeutic regimen comprising a PCSK9 inhibitor administered at a particular dosing frequency and amount, the daily dose of statin administered or prescribed to the patient may (a) stay the same, (b) increase, or (c) decrease (e.g., up-titrate or down-titrate) in comparison to the daily statin dose the patient was taking before starting the PCSK9 inhibitor therapeutic regimen, depending on the therapeutic needs of the patient.

### Therapeutic Efficacy

The methods of the present invention will result in the reduction in serum levels of one or more lipid components selected from the group consisting of LDL-C, ApoB100, non-HDL-C, total cholesterol, VLDL-C, triglycerides, Lp(a) and remnant cholesterol. For example, according to certain embodiments of the present invention, administration of a pharmaceutical composition comprising a PCSK9 inhibitor to a patient with primary hypercholesterolemia that is not adequately controlled by a stable daily moderate-dose therapeutic statin regimen, (e.g., administration of the PCSK9 inhibitor on top of the patient's moderate-dose statin therapy) will result in a mean percent reduction from baseline in serum low density lipoprotein cholesterol (LDL-C) of at least about 25%, 30%, 40%, 50%, 60%, or greater; a mean percent reduction from baseline in ApoB100 of at least about 25%, 30%, 40%, 50%, 60%, or greater; a mean percent reduction from baseline in non-HDL-C of at least about 25%, 30%, 40%, 50%, 60%, or greater; a mean percent reduction from baseline in total cholesterol of at least about 10%, 15%, 20%, 25%, 30%, 35%, or greater; a mean percent reduction from baseline in VLDL-C of at least about 5%, 10%, 15%, 20%, 25%, 30%, or greater; a mean percent reduction from baseline in triglycerides of at least about 5%, 10%, 15%, 20%, 25%, 30%, 35% or greater; and/or a mean percent reduction from baseline in Lp(a) of at least about 5%, 10%, 15%, 20%, 25%, or greater.

### PCSK9 Inhibitors

The methods of the present invention comprise administering to a patient a therapeutic composition comprising a PCSK9 inhibitor. As used herein, a “PCSK9 inhibitor” is any agent which binds to or interacts with human PCSK9 and inhibits the normal biological function of PCSK9 in vitro or in vivo. Non-limiting examples of categories of PCSK9 inhibitors include small molecule PCSK9 antagonists, peptide-based PCSK9 antagonists (e.g., “peptibody” molecules), and antibodies or antigen-binding fragments of antibodies that specifically bind human PCSK9.

The term “human proprotein convertase subtilisin/kexin type 9” or “human PCSK9” or “hPCSK9”, as used herein, refers to PCSK9 having the nucleic acid sequence shown in SEQ ID NO: 197 and the amino acid sequence of SEQ ID NO: 198, or a biologically active fragment thereof.

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-PCSK9 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3, (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PCSK9, as used in the context of the present invention, includes antibodies that bind PCSK9 or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PCSK9, however, have cross-reactivity to other antigens, such as PCSK9 molecules from other (non-human) species.

The anti-PCSK9 antibodies useful for the methods of the present invention may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present invention.

The present invention also includes methods involving the use of anti-PCSK9 antibodies comprising variants of any of the HCVR, LCVR, and/or CDR amino acid sequences disclosed herein having one or more conservative substitutions. For example, the present invention includes the use of anti-PCSK9 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

According to certain embodiments, the anti-PCSK9 antibody used in the methods of the present invention is an antibody with pH-dependent binding characteristics. As used herein, the expression “pH-dependent binding” means that the antibody or antigen-binding fragment thereof exhibits “reduced binding to PCSK9 at acidic pH as compared to neutral pH” (for purposes of the present disclosure, both expressions may be used interchangeably). For the example, antibodies “with pH-dependent binding characteristics” includes antibodies and antigen-binding fragments thereof that bind PCSK9 with higher affinity at neutral pH than at acidic pH. In certain embodiments, the antibodies and antigen-binding fragments of the present invention bind PCSK9 with at least 3, 5, 10, 15, 20, 25, 30, 35, 40, 45, 50, 55, 60, 65, 70, 75, 80, 85, 90, 95, 100, or more times higher affinity at neutral pH than at acidic pH.

According to this aspect of the invention, the anti-PCSK9 antibodies with pH-dependent binding characteristics may possess one or more amino acid variations relative to the parental anti-PCSK9 antibody. For example, an anti-PCSK9 antibody with pH-dependent binding characteristics may contain one or more histidine substitutions or insertions, e.g., in one or more CDRs of a parental anti-PCSK9 antibody. Thus, according to certain embodiments of the present invention, methods are provided comprising administering an anti-PCSK9 antibody which comprises CDR amino acid sequences (e.g., heavy and light chain CDRs) which are identical to the CDR amino acid sequences of a parental anti-PCSK9 antibody, except for the substitution of one or more amino acids of one or more CDRs of the parental antibody with a histidine residue. The anti-PCSK9 antibodies with pH-dependent binding may possess, e.g., 1, 2, 3, 4, 5, 6, 7, 8, 9, or more histidine substitutions, either within a single CDR of a parental antibody or distributed throughout multiple (e.g., 2, 3, 4, 5, or 6) CDRs of a parental anti-PCSK9 antibody. For example, the present invention includes the use of anti-PCSK9 antibodies with pH-dependent binding comprising one or more histidine substitutions in HCDR1, one or more histidine substitutions in HCDR2, one or more histidine substitutions in HCDR3, one or more histidine substitutions in LCDR1, one or more histidine substitutions in LCDR2, and/or one or more histidine substitutions in LCDR3, of a parental anti-PCSK9 antibody.

As used herein, the expression “acidic pH” means a pH of 6.0 or less (e.g., less than about 6.0, less than about 5.5, less than about 5.0, etc.). The expression “acidic pH” includes pH values of about 6.0, 5.95, 5.90, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

### Preparation of Human Antibodies

Methods for generating human antibodies in transgenic mice are known in the art. Any such known methods can be used in the context of the present invention to make human antibodies that specifically bind to human PCSK9.

Using VELOCIMMUNE™ technology (see, for example, U.S. Pat. No. 6,596,541, Regeneron Pharmaceuticals) or any other known method for generating monoclonal antibodies, high affinity chimeric antibodies to PCSK9 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc, using standard procedures known to those skilled in the art. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies that can be used in the methods of the present invention possess high affinities, as described above, when measured by binding to antigen either immobilized on solid phase or in solution phase. The mouse constant regions are replaced with desired human constant regions to generate the fully human antibodies of the invention. While the constant region selected may vary according to specific use, high affinity antigen-binding and target specificity characteristics reside in the variable region.

Specific examples of human antibodies or antigen-binding fragments of antibodies that specifically bind PCSK9 which can be used in the context of the methods of the present invention include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs: 1 and 11, or a substantially similar sequence thereof having at least 90%, at least 95%, at least 98% or at least 99% sequence identity. The antibody or antigen-binding fragment may comprise the three light chain CDRs (LCVR1, LCVR2, LCVR3) contained within a light chain variable region (LCVR) having an amino acid sequence selected from the group consisting of SEQ ID NOs: 6 and 15, or a substantially similar sequence thereof having at least 90%, at least 95%, at least 98% or at least 99% sequence identity.

In certain embodiments of the present invention, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs: 1/6 and 11/15.

In certain embodiments of the present invention, the anti-PCSK9 antibody, or antigen-binding fragment thereof, that can be used in the methods of the present invention has HCDR1/HCDR2/HCDR3/LCDR1/LCDR2/LCDR3 amino acid sequences selected from SEQ ID NOs: 2/3/4/7/8/10 (mAb316P) and 12/13/14/16/17/18 (mAb300N) (See US Patent App. Publ No. 2010/0166768).

In certain embodiments of the present invention, the antibody or antigen-binding fragment thereof comprises HCVR/LCVR amino acid sequence pairs selected from the group consisting of SEQ ID NOs: 1/6 and 11/15.

### Pharmaceutical Compositions and Methods of Administration

The present invention includes methods which comprise administering a PCSK9 inhibitor to a patient, wherein the PCSK9 inhibitor is contained within a pharmaceutical composition. The pharmaceutical compositions of the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of PCSK9 inhibitor (e.g., anti-PCSK9 antibody) administered to a subject according to the methods of the present invention is, generally, a therapeutically effective amount. As used herein, the phrase “therapeutically effective amount” means a dose of PCSK9 inhibitor that results in a detectable reduction (at least about 5%, 10%, 15%, 20%, 25%, 30%, 35%, 40%, 45%, 50%, 55%, 60%, 65%, 70%, 75%, or more from baseline) in one or more parameters selected from the group consisting of LDL-C, ApoB100, non-HDL-C, total cholesterol, VLDL-C, triglycerides, Lp(a) and remnant cholesterol.

In the case of an anti-PCSK9 antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-PCSK9 antibody. In certain embodiments, the therapeutically effective amount comprises about 75 mg of the anti-PCSK9 antibody (e.g., 70 mg, 71 mg, 72 mg, 73 mg, 74 mg, 75 mg, 76 mg, 77 mg, 78 mg, 79 mg, or 80 mg) of anti-PCSK9 antibody).

The amount of anti-PCSK9 antibody contained within the individual doses may be expressed in terms of milligrams of antibody per kilogram of patient body weight (i.e., mg/kg). For example, the anti-PCSK9 antibody may be administered to a patient at a dose of about 0.0001 to about 10 mg/kg of patient body weight.

### Combination Therapies

As described elsewhere herein, the methods of the present invention may comprise administering a PCSK9 inhibitor to a patient in combination with the patient's previously prescribed stable daily moderate-dose therapeutic statin regimen. According to certain embodiments of the present invention, additional therapeutic agents, besides a statin, may be administered to the patient in combination with the PCSK9 inhibitor. Examples of such additional therapeutic agents include e.g., (1) an agent which inhibits cholesterol uptake and or bile acid re-absorption (e.g., ezetimibe); (2) an agent which increase lipoprotein catabolism (such as niacin); and/or (3) activators of the LXR transcription factor that plays a role in cholesterol elimination such as 22-hydroxycholesterol.

### Administration Regimens

According to certain embodiments of the present invention, multiple doses of a PCSK9 inhibitor (i.e., a pharmaceutical composition comprising a PCSK9 inhibitor) may be administered to a subject over a defined time course (e.g., on top of a daily therapeutic statin regimen). The methods according to this aspect of the invention comprise sequentially administering to a subject multiple doses of a PCSK9 inhibitor. As used herein, “sequentially administering” means that each dose of PCSK9 inhibitor is administered to the subject at a different point in time, e.g., on different days separated by a predetermined interval (e.g., hours, days, weeks or months). The present invention includes methods which comprise sequentially administering to the patient a single initial dose of a PCSK9 inhibitor, followed by one or more secondary doses of the PCSK9 inhibitor, and optionally followed by one or more tertiary doses of the PCSK9 inhibitor.

The terms “initial dose,” “secondary doses,” and “tertiary doses,” refer to the temporal sequence of administration of the individual doses of a pharmaceutical composition comprising a PCSK9 inhibitor. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the PCSK9 inhibitor, but generally may differ from one another in terms of frequency of administration. In certain embodiments, however, the amount of PCSK9 inhibitor contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

According to exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of antigen-binding molecule which is administered to a patient prior to the administration of the very next dose in the sequence with no intervening doses.

The methods according to this aspect of the invention may comprise administering to a patient any number of secondary and/or tertiary doses of a PCSK9 inhibitor. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2, 4, 6, 8 or more weeks after the immediately preceding dose. Similarly, in embodiments involving multiple tertiary doses, each tertiary dose may be administered at the same frequency as the other tertiary doses. For example, each tertiary dose may be administered to the patient 1 to 2, 4, 6, 8 or more weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In certain embodiments, the anti-PCSK9 antibody is administered to a subject at a dose of about 50 mg every two weeks, for example for at least three doses.

In certain embodiments, the anti-PCSK9 antibody is administered to a subject at a dose of about 75 mg every two weeks, for example for at least three doses.

In certain embodiments, the anti-PCSK9 antibody is administered to a subject at a dose of about 150 mg every two weeks, for example for at least three doses.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Generation of Human Antibodies to Human PCSK9

Human anti-PCSK9 antibodies were generated as described in U.S. Pat. No. 8,062,640. The exemplary PCSK9 inhibitor used in the following Example is the human anti-PCSK9 antibody designated “mAb316P,” also known as “Alirocumab.” mAb316P has the following amino acid sequence characteristics: heavy chain variable region (HCVR) comprising SEQ ID NO:-1; light chain variable domain (LCVR) comprising SEQ ID NO: 6; heavy chain complementarity determining region 1 (HCDR1) comprising SEQ ID NO: 2; HCDR2 comprising SEQ ID NO: 3; HCDR3 comprising SEQ ID NO: 4; light chain complementarity determining region 1 (LCDR1) comprising SEQ ID NO: 7; LCDR2 comprising SEQ ID NO: 8; and LCDR3 comprising SEQ ID NO: 10.

### Example 2

### A Multicenter, Randomized, Double-Blind, Parallel-Group, Placebo-Controlled Study Evaluating the Efficacy and Safety of Three Doses of mAb316P Over 12 Weeks in Patients with Primary Hypercholesterolemia and LDL-Cholesterol ≧100 mg/dL (≧2.59 Mmol/L) on Ongoing Stable Atorvastatin Therapy

### Study Objectives

The primary objective of the present study was to evaluate the effect of mAb316P on low-density lipoprotein cholesterol (LDL-C) levels after 12 weeks of treatment in comparison with placebo in patients with LDL-C ≧100 mg/dL (≧2.59 mmol/L) on ongoing stable atorvastatin therapy. The secondary objectives were: to evaluate the effects of mAb316P on other lipid levels after 12 weeks of treatment in comparison with placebo, to evaluate the safety and tolerability of mAb316P, to evaluate the development of anti-mAb316P antibodies, and to evaluate the pharmacokinetics of mAb316P.

### Study Design

This was a multicenter, randomized, double-blind, parallel-group, placebo-controlled, 12-week study conducted in Japan to assess the efficacy and safety of mAb316P in patients with an elevated LDL-C (≧100 mg/dL) treated with stable dose of atorvastatin (5-20 mg) for at least 6 weeks. During the screening/run-in period, patients were stabilized to atorvastatin treatment (5-20 mg) for at least 6 weeks, if they were not already. The randomization was stratified based on the dose of atorvastatin (<10 mg, 210 mg) received prior to randomization. During the double-blind treatment period, patients returned to the site every 2 weeks to receive study treatment (mAb316P or placebo). The double-blind treatment period was then followed by an 8-week follow-up period.

This study consisted of 3 periods: screening, double-blind treatment, and follow-up.

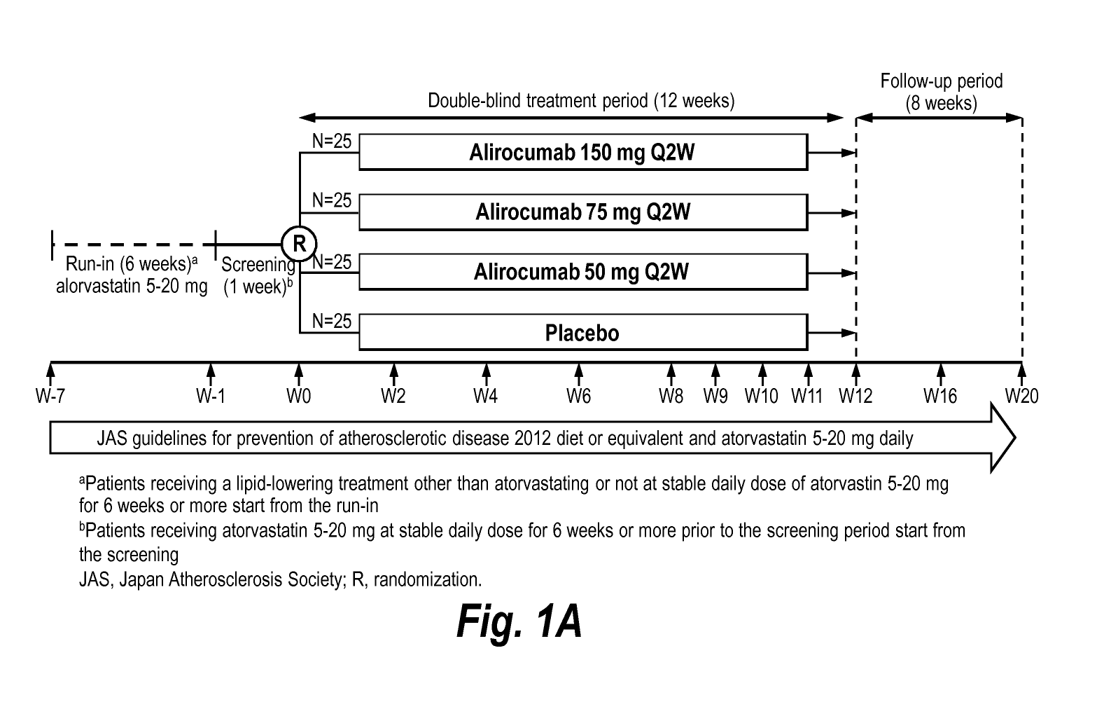

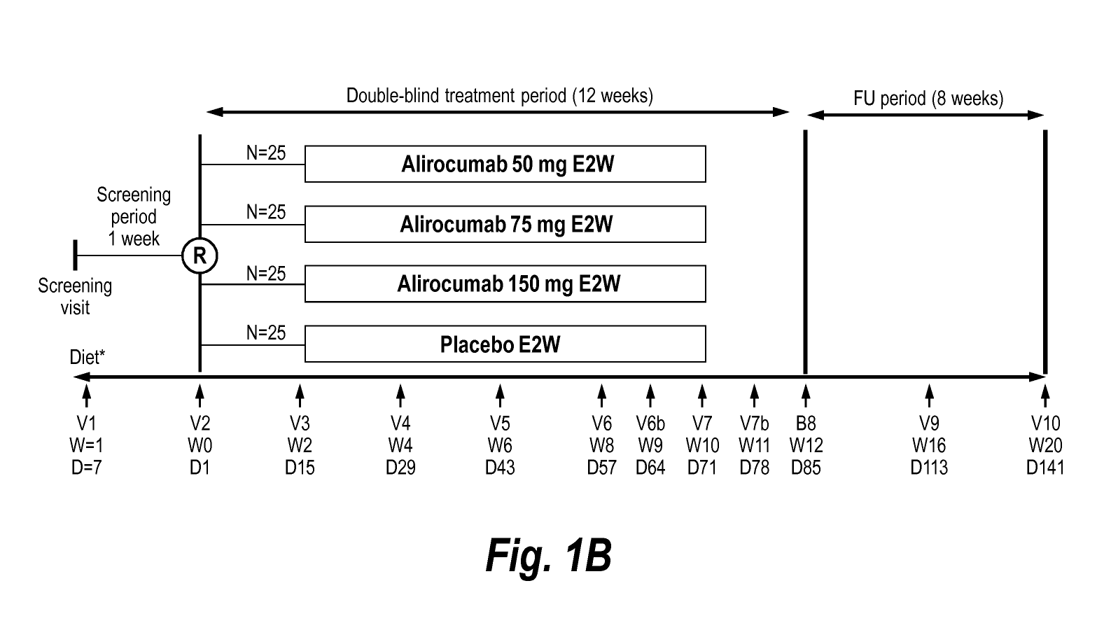

The screening period had 2 options, depending on the status of the patient at screening. Option 1 (FIG. 1A): for patients receiving atorvastatin 5-20 mg at stable dose for at least 6 weeks prior to screening, the screening period was 1 week. This screening period was started by a screening visit (Week-1, V1), during which patients were evaluated for eligibility to enter the double-blind treatment period. Option 2 (FIG. 1B): for patients receiving a lipid lowering treatment other than atorvastatin, or not at stable dose of atorvastatin 5-20 mg for 6 weeks prior to screening, or drug naive patients, the screening period included a run-in period on atorvastatin (5-20 mg) with: a pre-screening visit (Week-7, V1a) during which pre-eligibility assessments were performed to permit the entry of patients into the run-in period. If applicable, at this pre-screening visit (Week-7, V1a) patients were asked to stop any lipid lowering treatment previously received. The choice of atorvastatin dose was left to the investigator's discretion. Then after 6 weeks of run-in period, a screening visit (Week −1, V1) was held during which the full eligibility assessment was performed.

A randomized, double-blind, placebo-controlled, parallel-group treatment period of 12 weeks evaluating three dose regimens of mAb316P. The randomization was stratified by the dose of atorvastatin received prior to randomization (<10 mg, 210 mg).

A follow-up period of 8 weeks during which patients returned for follow up visits 4 and 8 weeks after the end of study treatment period (Week 12).

Thus, the duration of study participation depended on the status of the patient at screening. For patients receiving atorvastatin 5-20 mg at a stable dose for at least 6 weeks prior to screening, the study participation was approximately 21 weeks, including a screening period of 1 week, a double-blind treatment period of 12 weeks, and a follow-up period of 8 weeks. For patients receiving a lipid lowering treatment other than atorvastatin, or not at stable dose of atorvastatin 5-20 mg for at least 6 weeks prior to screening, or drug naive, the study participation was approximately 27 weeks, including a screening period of 7 weeks (including a run-in period of 6 weeks), a double-blind treatment period of 12 weeks, and a follow-up period of 8 weeks.

All patients were instructed to be on stable dose of atorvastatin as well as on a stable diet (Japan Atherosclerosis Society Guidelines for Prevention of Atherosclerotic Cardiovascular Diseases or equivalent) throughout the entire study duration from screening up to end of study.

### Selection of Patients

Patients meeting all of the following inclusion criteria were considered for enrollment into the study: patients with primary hypercholesterolemia treated with atorvastatin at stable dose of 5-20 mg for at least 6 weeks prior to screening and likely to have LDL-C ≧100 mg/dL (≧2.59 mmol/L) at the screening visit (Week −1), or patients with primary hypercholesterolemia who were receiving a lipid-lowering treatment other than atorvastatin, or who were not at stable dose of atorvastatin 5-20 mg for at least 6 weeks prior to screening and who are likely to have LDL-C ≧100 mg/dL (≧2.59 mmol/L) after the run-in period on atorvastatin therapy (Week −1).

Patients who met all of the above inclusion criteria were screened for the following exclusion criteria. Exclusion criteria related to study methodology were:

1) LDL-C<100 mg/dL (<2.59 mmol/L) at Week −1 (V1): at the first visit for patients who are being treated with stable dose of atorvastatin 5-20 mg for at least 6 weeks prior to screening period, or after the run-in period on atorvastatin (5-20 mg) for patients receiving a lipid lowering treatment other than atorvastatin, or not at stable dose of atorvastatin 5-20 mg for at least 6 weeks prior to the screening period;

2) patients aged <20 or >75 years at Week −7 or Week −1;

3) body mass index (BMI)<18 or >40 kg/m² at Week −7 or week −1;

4) patients not previously instructed on a cholesterol-lowering diet;

5) use of a statin other than atorvastatin 5-20 mg, or use of other lipid lowering drugs including but not limited to fibrates, bile acid sequestrants, niacin >500 mg/day, intestinal cholesterol absorption (ICA) blockers, red yeast rice or omega-3 fatty acids at doses >1000 mg during the screening period;

6) use of nutraceutical products that may affect lipids (e.g., omega-3 fatty acids at doses <1000 mg, plant stanols such as found in Benecol, flax seed oil, psyllium) not on stable dose for at least 6 weeks prior to screening period and/or during the screening period and/or not intended to be used at stable dose throughout the study;

7) patients with type 1 diabetes;

8) patients with type 2 diabetes treated with insulin at Week-7 or Week-1;

9) patients with type 2 diabetes and with an HbA1c≧8.5% (NGSP value) at Week −1 (considered poorly controlled);

10) presence of any clinically significant endocrine disease known to influence serum lipids or lipoproteins (Note: Patients on thyroid replacement therapy can be included if the dosage of thyroxine has been stable for at least 12 weeks prior to screening and their s-Thyroid Stimulating Hormone (s-TSH) level is within ±10% of the normal range of the Central Laboratory);

11) patients with uncontrolled blood pressure (>160/100 mmHg) at Week −7 or Week −1 or Week 0;

12) history of myocardial infarction, coronary artery bypass graft surgery (CABG), percutaneous coronary intervention (PCI), unstable angina pectoris, carotid surgery or stenting, cerebrovascular accident, or transient ischemic attack (TIA) within 6 months prior to the screening;

13) history of heart failure (NYHA Class II-IV) within 12 months prior to the screening;

14) history of ileal bypass, gastric bypass or other bariatric surgery within 12 months prior to screening, or if surgery took place more than 12 months prior to the screening, unstable weight (variation >5 kg) within 2 months prior to the screening;

15) presence or history of cancer within the past 5 years with the exception of adequately treated localized basal skin cancer;

16) known history of HIV positivity;

17) patients with a history (during the past 6 months) or concurrent DSM-IV substance abuse or dependence (excluding nicotine and caffeine);

18) conditions/situations such as: any clinically significant abnormality identified at the time of screening that in the judgment of the Investigator would preclude safe completion of the study or constrain endpoints assessment such as major systemic diseases, patients with short life expectancy; or patients considered by the Investigator as inappropriate for this study for any reason, e.g.: those deemed unable to meet specific protocol requirements, such as scheduled visits; those with likelihood of requiring treatment during the screening phase and treatment phase with drugs not permitted by the clinical study protocol; investigator, pharmacist, study coordinator, other study staff or relative thereof directly involved in the conduct of the protocol, etc; or presence of any other conditions (e.g. geographic, social . . . ) actual or anticipated, that the Investigator feels would restrict or limit the patient's participation for the duration of the study;

19) patient who has previously been treated with mAb316P or any other anti-PCSK9 in a clinical trial;

20) patient who has taken other investigational drugs within 1 month or 5 half lives, whichever is longer;

21) patient who withdraws consent during the screening period (patient who is not willing to continue or fails to return);

22) laboratory findings with fasting condition measured before randomization: positive test for hepatitis B surface antigen and/or hepatitis C antibody; triglycerides (TG) >350 mg/dL (>3.95 mmol/L) at Week −7 or Week −1; neutrophils <1,500/mm³ and/or platelets <100,000/mm³; positive serum or urine pregnancy test in females of childbearing potential; abnormal sensitive TSH level (>ULN or <LLN) according to the normal values of the Central Laboratory (for patients on thyroid replacement therapy, refer to exclusion criterion E 10) at Week-1; evidence of renal impairment as determined by: Men: serum creatinine >1.5×ULN, Women: serum creatinine >1.4×ULN; ALT or AST>3×ULN (1 repeat lab is allowed); CPK>3×ULN (1 repeat lab is allowed).

Exclusion criteria related to mandatory background therapies were: 1) all contraindications to the protocol mandated background therapy (i.e., atorvastatin) or warning/precaution of use (when appropriate) as displayed in the respective National Product Labeling that was used for defining these exclusion criteria. Exclusion criteria related to the current knowledge of mAb316P compound were: known hypersensitivity to monoclonal antibody therapeutics; pregnant or breast-feeding women; women of childbearing potential with no effective contraceptive method of birth control and/or who are unwilling or unable to be tested for pregnancy (Note: Women of childbearing potential must have a confirmed negative pregnancy test at screening and randomization visits. They must use an effective contraceptive method throughout the entire duration of the study treatment, and for 10 weeks after the last intake of investigational medicine product (IMP), and agree to repeat urine pregnancy test at designated visits. The applied methods of contraception have to meet the criteria for a highly effective method of birth control according to the “Note for guidance on nonclinical safety studies for the conduct of human clinical trials and marketing authorization for pharmaceuticals (CPMP/ICH/286/95)”. Postmenopausal women must be amenorrheic for at least 12 months).

### Study Treatments

Sterile mAb316P drug product was supplied at a concentration of 150 mg/mL in 10 mM histidine, pH 6.0, 0.2% (w/v) polysorbate 20, and 10% (w/v) sucrose. Drug product was supplied as a liquid with a 2 mL maximum withdrawable content in a nominal 5 mL glass vial.

Placebo for mAb316P was prepared in the same formulation as mAb316P without the addition of protein.

mAb316P was administered in SC injections of 1 mL each on Week 0 (V2), Week 2 (V3), Week 4 (V4), Week 6 (V5), Week 8 (V6), and Week 10 (V7). All study treatment injections were administered in the abdomen; administration to the extremities was not allowed as this may lead to different bioavailability absorption.

All patients received atorvastatin 5-20 mg as background therapy. The dose was stable for at least 6 weeks prior to screening visit (Week −1). Atorvastatin dose was recorded throughout the study. All patients were treated with atorvastatin, once daily in the evening with dinner, for the entire duration of the study, including the follow-up period. They continued to receive the same once daily dose 5-20 mg as they received prior to randomization.

### Dose Regimen

Three dose levels of mAb316P were given: 50 mg, 75 mg, and 150 mg. The doses were administered every two weeks (Q2W) at weeks 0, 2, 4, 6, 8, and 10. Placebo for mAb316P was also administered according to the same schedule.

Atorvastatin was administered in an open-label manner as a background therapy. Atorvastatin was used at stable dose throughout the study. Tablets of atorvastatin are provided in 5 mg and 10 mg. 1 or 2 tablets of atorvastatin was administered orally for 5 mg, 10 mg, 15 mg and 20 mg once daily.

### Concomitant Medication

A concomitant medication is any treatment received by the patient concomitantly to any IMP(s). Treatments in addition to the IMP (mAb316P) should be kept to a minimum during the study. However, if these were considered necessary for the patient's welfare and were unlikely to interfere with the IMP, they may be given at the discretion of the Investigator, with a stable dose (when possible).

The following drugs were not be permitted during the study, including the post-treatment follow up period: statin other than atorvastatin at the doses indicated in the protocol, and other lipid lowering drugs including but not limited to fibrates, bile acid sequestrants, niacin >500 mg, intestinal cholesterol absorption (ICA) blockers, red yeast rice or omega-3 fatty acids at doses >1000 mg; thyroid preparations or thyroxin treatment (except in patients on replacement therapy); and insulin treatment.

Women of childbearing potential must take an effective contraceptive method throughout the study treatment up to 10 weeks after the last IMP injection (i.e. V10, Follow-up visit).

Any therapy other than the prohibited concomitant therapy described above was allowed and was recorded. Nutraceutical products that may affect lipids (e.g., omega-3 fatty acids at doses <1000 mg, plant stanols such as found in Benecol, flax seed oil, psyllium) were allowed only if they have been used at a stable dose for at least 6 weeks prior to and during the screening period, and if they were maintained at a stable dose throughout the study.

### Study Endpoints

The primary efficacy endpoint was the percent change in calculated LDL-C from baseline to Week 12, which was defined as: 100×(calculated LDL-C value at Week 12−calculated LDL-C value at baseline)/calculated LDL-C value at baseline. The baseline calculated LDL-C value was the average of the LDL-C levels obtained from Week −1 and up to before the first IMP injection. The calculated LDL-C at Week 12 was the LDL-C level obtained during the efficacy period and, within or before the Week 12 time window (Day 78 to 105). The efficacy period was defined as the time from the first IMP injection up to 21 days after the last IMP injection. All calculated LDL-C values (scheduled or unscheduled, fasting or not fasting) may be used to provide a value for the primary efficacy endpoint if appropriate according to above definition.

The secondary endpoints of the present study were: 1) absolute change in calculated LDL-C from baseline to Week 12; 2) proportion of patients achieving an LDL-C level lower than 100 mg/dL (2.59 mmol/L) at Week 12; 3) proportion of patients achieving an LDL-C level lower than 70 mg/dL (1.81 mmol/L) at Week 12; 4) percent change in TC, HDL-C, TG, non HDL-C, Apo B, Apo A1 and Lp(a) from baseline to Week 12; and 5) absolute change in TC, HDL-C, TG, non HDL-C, the ratio Apo B/Apo A1 from baseline (Week 0) to Week 12.

The safety endpoints included adverse events and serious adverse events. Vital signs, ECG, hematology, and serum chemistry were also monitored.

Other endpoints were: 1) Anti-mAb316P anti-drug-antibody status (positive/negative) and titres; 2) pharmacokinetics assessment; and, optionally, 3) pharmacogenetic associations.

### Study Procedures

The patient was in fasting conditions (i.e., overnight, at least 10-12 hours fast and refrain from smoking) for all visits from Week −7 to the final visit of the follow-up period Week 20 (V10). Alcohol consumption within 48 hours and intense physical exercise within 24 hours preceding the blood sampling were discouraged.

Body weight was obtained with the patient wearing undergarments or very light clothing and no shoes, and with an empty bladder. The same scale was used throughout the study. The use of calibrated balance scales was recommended, if possible. Self-reported weights were not acceptable; patients must not read the scales themselves.

Height was measured, as self-reported heights were not acceptable.

Blood pressure (BP) was measured in supine position under standardized conditions, approximately at the same time of the day, on the same arm, with the same apparatus (after the patient has rested comfortably in sitting position for at least five minutes). Both systolic blood pressure (SBP) and diastolic blood pressure (DBP) were recorded.

Heart rate was measured at the time of the measurement of blood pressure.

The 12-lead ECGs were performed after at least 10 minutes rest and in the supine position. The electrodes were positioned at the same place as much as possible, for each ECG recording throughout the study.

The blood sampling for determination of lipid parameters [i.e. TC, LDL-C, HDL-C, TG, non-HDL-C, Apo B, Apo A-1, ratio Apo B/Apo A-1, Lp(a)] and other blood parameters (HbA1c, fasting plasma glucose, hs-CRP) were performed in the morning, in fasting condition (i.e. overnight, at least 10-12 hours fast and refrain from smoking). Alcohol consumption within 48 hours and intense physical exercise within 24 hours preceding the blood sampling were discouraged. If the patient was not in fasting conditions, the blood sample was not be collected and a new appointment was given the day after (or as close as possible to this date) to the patient with instruction to be fasted.

The laboratory data was collected before the dose of study drug was administered.

Plasma samples for exploratory research were collected for exploratory research that may include the study of PCSK9 function, effect(s) of PCSK9 inhibition with a monoclonal antibody, and mechanisms of hyperlipidemia and heart disease. If needed, samples were also used to identify markers associated with toxicity.

### Analysis Populations

The efficacy population was a modified intent-to-treat (mITT) population, defined as randomized patients that had at least one calculated LDL-C from Week −1 up to before first IMP, and at least one evaluable calculated LDL-C during the time from first IMP up to 21 days after last IMP, and within or before the Week 12 time window (Day 78 to 105). The mITT population had 25 patients in each arm: placebo (25 patients), 50 mg alirocumab Q2W (25 patients), 75 mg alirocumab Q2W (25 patients), and 150 mg alirocumab Q2W (25 patients).

The safety population was defined as randomized patients who received at least one IMP injection. The safety population was analyzed according to actual treatment received. The safety population also had 25 patients in each arm: placebo (25 patients), 50 mg alirocumab Q2W (25 patients), 75 mg alirocumab Q2W (25 patients), and 150 mg alirocumab Q2W (25 patients).

### Results

A total of 100 patients were randomized to receive either placebo (25 patients) or alirocumab as 50 mg Q2W (25 patients), 75 mg Q2W (25 patients), or 150 mg Q2W (25 patients). Demographic and disease characteristics were generally consistent among the treatment groups, and mean baseline values for LDL-C ranged between 120.5 and 122.2 mg/dL across treatment groups. Baseline characteristics of the alirocumab and placebo groups are set forth in Table 1.

TABLE 1
Baseline characteristics of the alirocumab and placebo groups

| | | Alirocumab | Alirocumab | Alirocumab |
|---|---|---|---|---|
| | Placebo | 50 mg Q2W | 75 mg Q2W | 150 mg Q2W |
| | (n = 25) | (n = 25) | (n = 25) | (n = 25) |
| Age, years, mean (SD) | 58.6 (9.2) | 57.8 (12.3) | 56.3 (12.0) | 58.2 (8.8) |
| Female, n (%) | 11 (44) | 16 (64) | 12 (48) | 16 (64) |
| BMI, kg/m2, mean (SD) | 25.8 (3.2) | 24.3 (3.9) | 24.5 (3.3) | 24.2 (4.4) |
| Any medical/surgical | 24 (96) | 25 (100) | 23 (92) | 23 (92) |
| history^{a}, n (%) | | | | |
| Hypertension | 13 (52) | 9 (36) | 8 (32) | 5 (20) |
| Type 2 diabetes | 4 (16) | 5 (20) | 2 (8) | 5 (20) |
| Cerebrovascular disease | 2 (8) | 2 (8) | 1 (4) | 0 (0) |
| Peripheral vascular disease | 1 (4) | 1 (4) | 0 (0) | 0 (0) |
| Coronary artery disease | 0 (0) | 1 (4) | 0 (0) | 0 (0) |

^{a}Medical/surgical history other than disease being treated in this study. Patients can be counted in several categories.
BMI = Body Mass Index.
SD = Standard Deviation

In this study, alirocumab was generally well-tolerated and met its primary efficacy endpoint, reducing LDL-C at week 12 significantly versus placebo: −2.7% for placebo vs. −54.8% for 50 mg, −62.3% for 75 mg and −71.1% for 150 mg (Table 2). At week 12, the percent decrease from baseline in LDL-C was significantly greater in all alirocumab groups compared to the placebo group, with a clear dose-response pattern. All patients (100%) in each of the alirocumab groups achieved an LDL-C level of <100 mg/dL compared with 8% of patients in the placebo group.

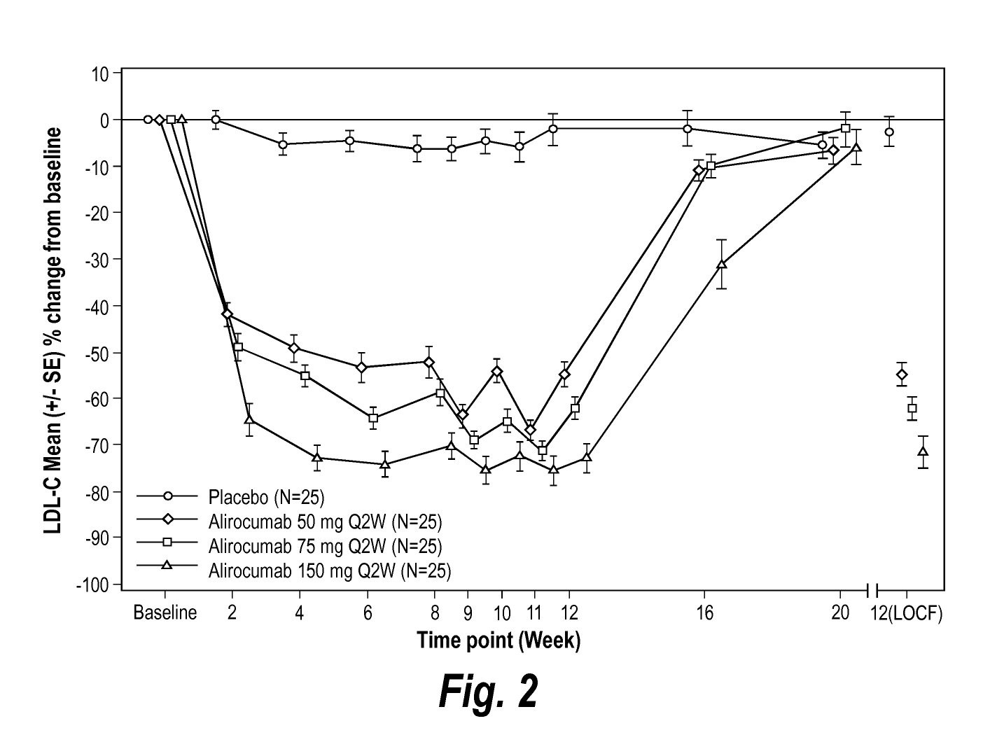

Alirocumab demonstrated a dose-dependent increase in the number of patients achieving ≧50% reduction from baseline in LDL-C. See FIG. 2 and Tables 2-5. By Week 12, significantly more patients in all alirocumab treatment arms reduced their LDL-C from baseline compared to placebo (>50% vs. 3%), which was the primary efficacy endpoint. Significant improvements were seen in all alirocumab groups by Week 2, and by Week 12 most had achieved an LDL-C goal <100 mg/dL (92% 150 mg arm; 80% 75 mg arm; 60% 50 mg arm; 0% placebo arm).

Table 2 below shows the primary analysis for the primary efficacy endpoint LDL-C (mg/dL) at week 12—LOCF analysis of covariance (mITT population). Table 3 shows the proportion of patients at week 12—LOCF achieving LDL-C<100 mg/dL, and achieving ≧50% reduction (mITT population). Table 4 shows selected lipid parameters at week 12—LOCF analysis of covariance. Table 5 shows selected lipid parameters at week 12—LOCF rank analysis of covariance.

TABLE 2
Primary efficacy endpoint LDL-C (mg/dL) at week 12 (mITT population)

| LDL Cholesterol | | 50 mg mAb316P | 75 mg mAb316P | 150 mg mAb316P |
|---|---|---|---|---|
| (mg/dL) | Placebo (N = 25) | Q2W (N = 25) | Q2W (N = 25) | Q2W (N = 25) |
| Baseline | | | | |
| Number | 25 | 25 | 25 | 25 |
| Mean (SD) | 121.0 (21.1) | 122.2 (16.6) | 120.9 (16.7) | 120.5 (16.2) |
| Median | 113.0 | 122.5 | 116.0 | 120.0 |
| Q1:Q3 | 107.5:131.0 | 113.0:134.5 | 111.5:128.5 | 109.0:130.5 |
| Min:Max | 89:184 | 95:151 | 98:169 | 93:148 |
| Week 12-LOCF | | | | |
| Number | 25 | 25 | 25 | 25 |
| Mean (SD) | 116.0 (16.8) | 54.4 (16.3) | 46.4 (18.5) | 35.0 (24.1) |
| Median | 117.0 | 57.0 | 43.0 | 30.0 |
| Q1:Q3 | 105.0:128.0 | 46.0:64.0 | 33.0:57.0 | 19.0:47.0 |
| Min:Max | 74:152 | 19:82 | 15:84 | 2:87 |
| Week 12-LOCF | | | | |
| % change from | | | | |
| baseline | | | | |
| Number | 25 | 25 | 25 | 25 |
| Mean (SD) | −2.55 (16.39) | −54.84 (13.82) | −62.12 (12.59) | −71.54 (17.70) |
| Median | −2.91 | −52.65 | −65.30 | −72.13 |
| Q1:Q3 | −15.00:8.04 | −62.60:−45.51 | −70.51:−53.85 | −82.81:−58.65 |
| Min:Max | −29.3:28.8 | −85.2:−30.2 | −86.8:−39.2 | −98.3:−30.4 |
| LS Mean (SE) | −2.67 (3.09) | −54.83 (3.09) | −62.25 (3.09) | −71.72 (3.09) |
| LS Mean Difference | | −52.16 (4.32) | −59.57 (4.32) | −69.05 (4.32) |
| (SE) vs placebo | | | | |
| 95% CI vs placebo | | (−60.75 to −43.58) | (−68.16 to −50.99) | (−77.64 to −60.47) |
| p-value vs placebo^{a} | | <0.0001* | <0.0001* | <0.0001* |

LOCF is Last Observation Carried Forward.
Least-squares (LS) means and p-values come from analysis of covariance (ANCOVA) with treatment group and randomization strata of atorvastatin dose (<10 mg, ≧10 mg) as fixed effects and baseline as covariate.
^{a}p values are not adjusted for multiplicity and provided for descriptive purpose only.
*indicates a statistically significant p value according to the hierarchal procedure.

Proportion of patients at week 12 achieving LDL-C <100 mg/dL and
≧50 reduction (mITT population)

| | | 50 mg mAb316P | 75 mg mAb316P | 150 mg mAb316P |
|---|---|---|---|---|
| | Placebo (N = 25) | Q2W (N = 25) | Q2W (N = 25) | Q2W (N = 25) |
| Week 12-LOCF | | | | |
| Number | 25 | 25 | 25 | 25 |
| <100 mg/dL | 2 (8.0%) | 25 (100%) | 25 (100%) | 25 (100%) |
| ≧100 mg/dL | 23 (92.0%) | 0 | 0 | 0 |
| Number | 25 | 25 | 25 | 25 |
| ≧50% of decrease | 0 | 15 (60.0%) | 20 (80.0%) | 23 (92.0%) |
| <50% of decrease | 25 (100%) | 10 (40.0%) | 5 (20.0%) | 2 (8.0%) |

TABLE 4
Selected lipid parameters at week 12.

| % change from | | | | |
|---|---|---|---|---|
| baseline to week | | 50 mg mAb316P | 75 mg mAb316P | 150 mg mAb316P |
| 12-LOCF | Placebo (N = 25) | Q2W (N = 25) | Q2W (N = 25) | Q2W (N = 25) |
| Total-C (mg/dL) | | | | |
| Number | 25 | 25 | 25 | 25 |
| LS Mean (SE) | −1.20 (2.11) | −31.88 (2.11) | −36.33 (2.11) | −41.40 (2.12) |
| LS Mean | | −30.68 (2.95) | −35.13 (2.96) | −40.20 (2.98) |
| Difference (SE) vs | | | | |
| placebo | | | | |
| 95% CI vs | | (−36.54 to −24.82) | (−41.01 to −29.26) | (−46.11 to −34.28) |
| placebo | | | | |
| p-value vs | | <0.0001 | <0.0001 | <0.0001 |
| placebo^{a} | | | | |
| HDL-C (mg/dL) | | | | |
| Number | 25 | 25 | 25 | 25 |
| LS Mean (SE) | −0.17 (2.04) | 5.09 (2.00) | 5.01 (1.99) | 3.22 (2.06) |
| LS Mean | | 5.26 (2.79) | 5.17 (2.81) | 3.39 (2.95) |
| Difference (SE) vs | | | | |
| placebo | | | | |
| 95% CI vs | | (−0.29 to 10.80) | (−0.41 to 10.76) | (−2.46 to 9.24) |
| placebo | | | | |
| p-value vs | | 0.0628 | 0.0692 | 0.2528 |
| placebo^{a} | | | | |

LOCF is Last Observation Carried Forward.
Least-squares (LS) means and p-values come from analysis of covariance (ANCOVA) with treatment group and randomization strata of atorvastatin dose (<10 mg, ≧10 mg) as fixed effects and baseline as covariate.
^{a}p values are not adjusted for multiplicity and provided for descriptive purpose only.

TABLE 5
Selected lipid parameters at week 12

| % change from | | | | |
|---|---|---|---|---|
| baseline to week | | 50 mg mAb316P | 75 mg mAb316P | 150 mg mAb316P |
| 12-LOCF | Placebo (N = 25) | Q2W (N = 25) | Q2W (N = 25) | Q2W (N = 25) |
| Triglycerides | | | | |
| (mg/dL) | | | | |
| Number | 25 | 25 | 25 | 25 |
| Mean (SD) | 5.19 (26.62) | −5.70 (35.29) | −11.11 (28.02) | −11.54 (20.13) |
| Median | 1.32 | −21.05 | −10.69 | −14.95 |
| Q1:Q3 | −13.10:22.49 | −28.19:4.17 | −25.00:6.51 | −24.14:0.00 |
| Min:Max | −54.2:53.7 | −38.2:111.0 | −58.9:72.4 | −46.0:38.0 |
| Effect size est. | | −17.19 | −16.89 | −19.35 |
| 95% CI | | (−29.39 to 2.71) | (−34.69 to -0.39) | (−37.45 to -4.65) |
| p-value vs | | 0.0629 | 0.0219 | 0.0059 |
| placebo | | | | |
| Lipoprotein (a) | | | | |
| (mg/dL) | | | | |
| Number | 25 | 25 | 25 | 25 |
| Mean (SD) | −3.92 (40.39) | −32.39 (26.60) | −42.35 (23.36) | −42.20 (30.40) |
| Median | −3.73 | −35.61 | −40.22 | −43.28 |
| Q1:Q3 | −26.20:8.43 | −52.53:−7.54 | −62.93:−27.37 | −69.44:-14.24 |
| Min:Max | −60.6:137.0 | −70.8:19.2 | −73.0:0.0 | −90.7:0.0 |
| Effect size est. | | −28.13 | −37.50 | −34.08 |
| 95% CI | | (−46.53 to −5.53) | (−55.00 to −18.00) | (−59.08 to −8.58) |
| p-value vs | | 0.0064 | 0.0003 | 0.0009 |
| placebo | | | | |

LOCF is Last Observation Carried Forward.

Each p-value comes from a rank analysis of covariance including terms for treatment, randomization strata of atorvastatin dose, and baseline value. The treatment term has two levels: the considered alirocumab dose and placebo. Effect size estimates and 95% Cls are built via the test statistic associated to each comparison. p values are not adjusted for multiplicity and provided for descriptive purpose only.

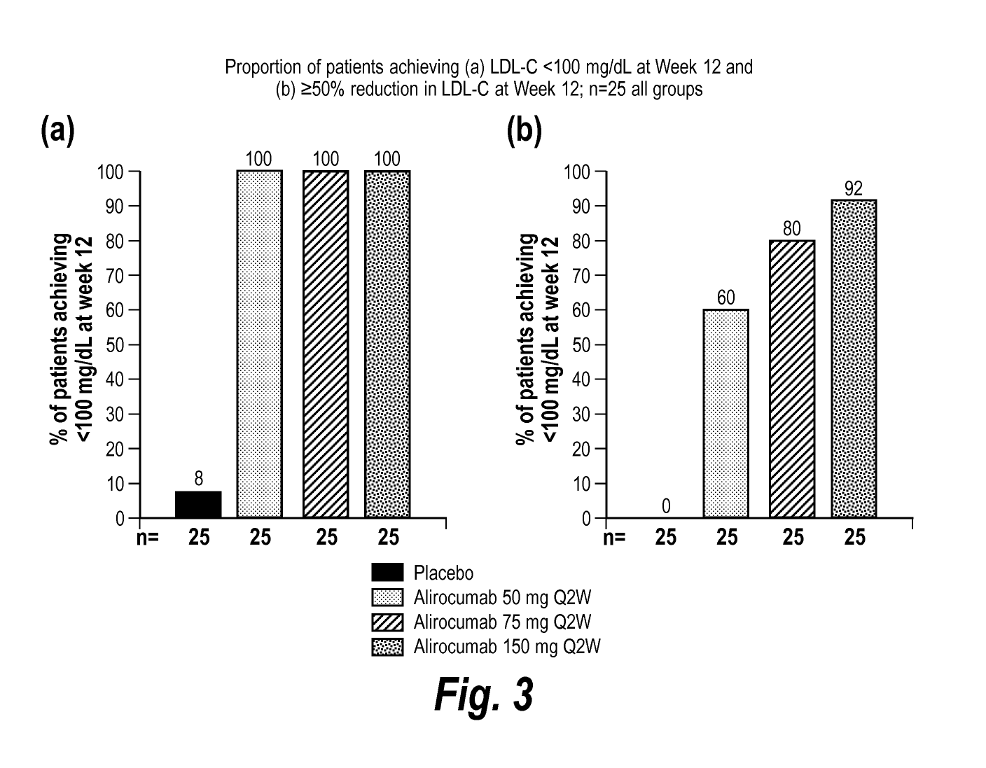

A summary of the LDL-C and other selected lipid parameters at baseline and Week 12 is set forth in Table 6. The mean calculated LDL-C at baseline was ˜120 mg/dL in each of the groups with an overall mean of 121.2 (17.5) mg/dL; with other baseline lipid parameters similar across the four groups (Table 6). At Week 12, alirocumab reduced LDL-C by 55-72% from baseline (an overall reduction of 62%) on background stable statin therapy (all P<0.0001) (see Table 2 and FIG. 2). Similarly, significant reductions were observed with alirocumab for total cholesterol and Lp(a), with greater reductions observed at higher doses (see Table 6). Trends were observed for HDL-C increases and triglyceride decreases (which were significant at higher doses) with alirocumab relative to placebo (see Table 2). All alirocumab-treated patients achieved LDL-C<100 mg/dL at Week 12 compared with just two placebo-treated patients (see FIG. 3). At the higher alirocumab doses of 75 and 150 mg Q2W, 80% and 92% of patients, respectively, achieved ≧50% reduction in LDL-C (see FIG. 3).

TABLE 6
LDL-C and other selected lipid parameters at baseline and Week 12

| | | Alirocumab | Alirocumab | Alirocumab |
|---|---|---|---|---|
| | Placebo | 50 mg Q2W | 75 mg Q2W | 150 mg Q2W |
| | (n = 25) | (n = 25) | (n = 25) | (n = 25) |
| Calculated LDL-C Baseline | 121.0 (21.1) | 122.2 (16.6) | 120.9 (16.7) | 120.5 (16.2) |
| (mg/dL)^{a} | | | | |
| Week 12, LOCF (mg/dL)^{a} % | 116.0 (16.8) | 54.4 (16.3) | 46.4 (18.5) | 35.0 (24.1) |
| change from baseline at | | | | |
| Week 12, LOCF^{b} | −2.7 (3.1) | −54.8 (3.1) | −62.3 (3.1) | −71.7 (3.1) |
| P-value vs placebo^{c} | | 0.0001 | 0.0001 | 0.0001 |
| Total cholesterol Baseline | 203.6 (27.7) | 205.2 (19.9) | 209.2 (24.3) | 212.5 (22.7) |
| (mg/dL)^{a} | | | | |
| Week 12, LOCF (mg/dL)^{a} | 200.5 (20.5) | 139.7 (22.2) | 133.2 (23.3) | 123.7 (28.3) |
| % change from baseline at | | | | |
| Week 12, LOCF^{b} | −1.2 (2.1) | −31.9 (2.1) | −36.3 (2.1) | −41.4 (2.1) |
| P-value vs placebo^{c} | | <0.0001 | <0.0001 | <0.0001 |
| HDL-C | 55.3 (11.2) | 58.8 (12.4) | 61.1 (15.3) | 68.1 (13.5) |
| Baseline (mg/dL)^{a} | | | | |
| Week 12, LOCF (mg/dL)^{a} | 56.4 (12.4) | 62.3 (13.5) | 63.9 (15.0) | 68.7 (12.5) |
| % change from baseline at | | | | |
| Week 12, LOCF^{b} | −0.2 (2.0) | 5.1 (2.0) | 5.0 (2.0) | 3.2 (2.1) |
| P-value vs placebo^{c} | | 0.0628 | 0.0692 | 0.2528 |
| Triglycerides Baseline | 128.0 | 126.0 | 119.5 | 107.0 |
| (mg/dL)^{a} | (101.5:156.5) | (90.5:136.5) | (86.5:182.5) | (87.0:124.5) |
| Week 12, LOCF (mg/dL)^{a} | 136.0 | 97.0 | 94.0 | 98.0 |
| % change from baseline at | (105.0:169.0) | (70.0:139.0) | (75.0:152.0) | (72.0:115.0) |
| Week 12, LOCF^{b} | 1.3 | −21.1 | −10.7 | −15.0 |
| | (−13.1:22.5) | (−28.2:4.2) | (−25.0:6.5) | (−24.1:0.0) |
| P-value vs placebo^{d} | | 0.0629 | 0.0219 | 0.0059 |
| Lp(a) | 11.7 | 9.6 | 14.8 | 16.9 |
| Baseline (mg/dL)a | (6.9:26.8) | (6.7:23.4) | (7.5:28.9) | (7.3:39.5) |
| Week 12, LOCF (mg/dL)a | 13.6 | 7.3 | 7.1 | 10.0 |
| % change from baseline at | (4.9:23.1) | (2.0:14.8) | (4.0:15.2) | (2.0:23.9) |
| Week 12, LOCFb | −3.7 | −35.6 | −40.2 | −43.3 |
| | (−26.2:8.4) | (−52.5:−7.5) | (−62.9:−27.4) | (−69.4:−14.2) |
| P-value vs placebo^{d} | | 0.0064 | 0.0003 | 0.0009 |

^{a}Values are mean (SD) for LDL-C, total cholesterol, and HDL-C, and median (Q1:Q3) for triglycerides and Lp(a).
^{b}Week 12-LOCF % changes are shown as least-squares mean (SE) for LDL-C, total cholesterol, and HDL-C, and median (Q1:Q3) for triglycerides and Lp(a).
^{c}P-values are from an ANCOVA model including treatment group and randomization strata of atorvastatin as fixed effects, and corresponding baseline value as covariate, and are not adjusted for multiplicity and are provided for descriptive purposes only.
^{d}P-values are from rank ANCOVA including terms for treatment, randomization strata of atorvastatin dose, and corresponding baseline value, and are not adjusted for multiplicity. They are provided for descriptive purposes only.
ANCOVA = analysis of covariance;
LOCF = last observation carried forward.

Treatment emergent adverse events (TEAEs) were reported in 8 (32%), 13 (52%), 12 (48%), and 16 (64%) patients in the placebo, alirocumab 50, 75, and 150 mg Q2W groups, respectively. The most frequently reported TEAEs occurred in the following system-organ-class (SOC), irrespective of the treatment group: infections and infestations, musculoskeletal and connective tissue disorders, and general disorders and administration site conditions. A summary of TEAEs and laboratory parameters are set forth in Table 7.

Two patients experienced serious adverse events (SAEs): vertigo in one patient in the placebo group, and breast cancer in one patient in the 150 mg Q2W group.

An adverse event of special interest (AESI) was reported in one patient in the 50 mg Q2W group, which was an increase in alanine aminotransferase (ALT).

No specific safety signal was seen in patients with levels of LDL-C<25 mg/dL (a total of 15 patients in alirocumab groups). A total of 8 patients reported local injection site reactions of mild intensity with no dose relationship.

TABLE 7
TEAEs and laboratory parameters

| | | Alirocumab | Alirocumab | Alirocumab |
|---|---|---|---|---|
| | Placebo | 50 mg Q2W | 75 mg Q2W | 150 mg Q2W |
| n (%) | (n = 25) | (n = 25) | (n = 25) | (n = 25 |
| Patients with any TEAE | 8 (32) | 13 (52) | 12 (48) | 16 (64) |
| Patients with any treatment- | 1 (4) | 0 | 0 | 1 (4) |
| emergent SAE | | | | |
| Patients with any TEAE | 0 | 0 | 0 | 0 |
| leading to death | | | | |

Primary System Organ Class
Preferred term (occurring >1 patient in an group

| | | | | |
|---|---|---|---|---|
| Infections and infestations | 5 (20) | 8 (32) | 8 (32) | 7 (28) |
| Nasopharyngitis | 2 | 6 (24) | 6 (24) | 4 (16) |
| Cystitis | 1 | 0 (0) | 0 (0) | 2 (8) |
| Musculoskeletal and | 1 | 4 (16) | 3(12) | 3 (12) |
| connective tissue disorders | | | | |
| Back pain | 1 | 2 (8) | 0 (0) | 1 (4) |
| General disorders and | 1 | 3 (12) | 2 (8) | 3 (12) |
| administration site conditions | | | | |
| Injection site reaction | 1 | 3 (12) | 2 (8) | 2 (8) |
| Injury, poisoning and | 1 | 0 (0) | 0 (0) | 2 (8) |
| procedural complications | | | | |
| Ligament sprain | 1 | 0 (0) | 0 (0) | 2 (8) |

Laboratory parameters

| | | | | |
|---|---|---|---|---|
| Alanine amino- | 0 (0) | 1 (4) | 0 (0) | 0 (0) |
| transferase >3 ULN | | | | |
| Aspartate amino- | 0 (0) | 1 (4) | 0 (0) | 0 (0) |
| transferase >3 ULN | | | | |
| Alkaline phos- | 0 (0) | 0 (0) | 1 (4) | 0 (0) |
| phatase >1.5 ULN | | | | |
| Creatine phos- | 0 (0) | 0 (0) | 1 (4) | 1 (4) |
| phokinase >3 ULN | | | | |

TEAE = treatment-emergent adverse event;
SAE = serious adverse event;
ULN = upper limit of normal

### Results Summary

This study demonstrated that alirocumab significantly reduced LDL-C in patients with hypercholesterolemia and was well tolerated at all tested doses. Specifically, alirocumab demonstrated robust efficacy over 12 weeks, reducing LDL-C from baseline by 55-72% in a dose-dependent manner when added to stable statin therapy (−2.7% for placebo vs. −54.8% for 50 mg, −62.3% for 75 mg and −71.1% for 150 mg). All patients in each of the alirocumab groups achieved an LDL-C level of <100 mg/dL compared with 8% of patients in the placebo group. TEAEs were reported in 49% of patients overall, the most frequent across the groups being nasopharyngitis. This study provides the first data for the alirocumab 75 mg Q2W dose on stable statin therapy in a Japanese population, reducing LDL-C by 62% from baseline.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method for treating hypercholesterolemia in a subject in need thereof, comprising:
a) selecting a subject who is currently being treated for hypercholesterolemia by administration of a background lipid-modifying therapy;
b) continuing treating the subject with the background lipid-modifying therapy; and
c) administering to the subject a pharmaceutical composition comprising about 75 mg of an antibody or antigen binding fragment thereof that specifically binds PCSK9 at a frequency of about once every other week (q2w), thereby treating the hypercholesterolemia in the subject.

2. The method of claim 1, wherein the method lowers the subject's low density lipoprotein-C (LDL-C) level by at least 50%.

3. The method of claim 1, wherein the method lowers the subject's low density lipoprotein-C (LDL-C) level by at least 60%.

4. The method of claim 1, wherein the subject, prior to or at the time of administration of the pharmaceutical composition, exhibits hypercholesterolemia defined as a serum low-density lipoprotein cholesterol (LDL-C) level of greater than about 70 mg/dL.

5. The method of claim 1, wherein the subject, prior to or at the time of administration of the pharmaceutical composition, exhibits hypercholesterolemia defined as a serum low-density lipoprotein cholesterol (LDL-C) level of greater than about 100 mg/dL.

6. The method of claim 1, wherein the subject has heterozygous Familial Hypercholesterolemia (heFH).

7. The method of claim 1, wherein the subject has a form of Hypercholesterolemia that is not Familial Hypercholesterolemia (nonFH).

8. The method of claim 1, wherein the background lipid-modifying therapy comprises a therapeutic agent selected from the group consisting of a statin, ezetimibe, a fibrate, niacin, an omega-3 fatty acid, and a bile acid resin.

9. The method of claim 1, wherein the background lipid-modifying therapy comprises a statin selected from the group consisting of cerivastatin, atorvastatin, simvastatin, pitavastatin, rosuvastatin, fluvastatin, lovastatin and pravastatin.

10. The method of claim 1, wherein the pharmaceutical composition is administered for at least 24 weeks.

11. The method of claim 1, wherein the pharmaceutical composition is administered subcutaneously.

12. The method of claim 1, wherein the antibody or antigen binding fragment thereof comprises the heavy and light chain CDRs of a HCVR/LCVR amino acid sequence pair selected from the group consisting of SEQ ID NOs: 1/6 and 11/15.

13. The method of claim 1, wherein the antibody or antigen-binding fragment thereof comprises heavy and light chain CDR amino acid sequences having SEQ ID NOs: 12, 13, 14, 16, 17, and 18.

14. The method of claim 1, wherein the antibody or antigen-binding fragment thereof comprises an HCVR having the amino acid sequence of SEQ ID NO: 11 and an LCVR having the amino acid sequence of SEQ ID NO: 15.

15. The method of claim 1, wherein the antibody or antigen-binding fragment thereof comprises heavy and light chain CDR amino acid sequences having SEQ ID NOs: 2, 3, 4, 7, 8, and 10.

16. The method of claim 1, wherein the antibody or antigen-binding fragment thereof comprises an HCVR having the amino acid sequence of SEQ ID NO: 1 and an LCVR having the amino acid sequence of SEQ ID NO: 6.

17. The method of claim 1, wherein the antibody or antigen-binding fragment thereof binds to the same epitope on PCSK9 as an antibody comprising heavy and light chain CDR amino acid sequences having SEQ ID NOs: 12, 13, 14, 16, 17, and 18; or SEQ ID NOs: 2, 3, 4, 7, 8, and 10.

18. The method of claim 1, wherein the antibody or antigen-binding fragment thereof competes for binding to PCSK9 with an antibody comprising heavy and light chain CDR amino acid sequences having SEQ ID NOs: 12, 13, 14, 16, 17, and 18; or SEQ ID NOs: 2, 3, 4, 7, 8, and 10.

19. A method for reducing low density lipoprotein-C (LDL-C) in a subject in need thereof, comprising:
a) selecting a subject who is currently being treated for hypercholesterolemia by administration of a background lipid-modifying therapy;
b) continuing treating the subject with the background lipid-modifying therapy; and
c) administering to the subject a pharmaceutical composition comprising about 75 mg of an antibody or antigen binding fragment thereof that specifically binds PCSK9 at a frequency of about once every other week (q2w), thereby reducing the LDL-C in the subject.

20. The method of claim 19, wherein the method lowers the subject's low density lipoprotein-C (LDL-C) level by at least 50%.

21. The method of claim 19, wherein the method lowers the subject's low density lipoprotein-C (LDL-C) level by at least 60%.

22. The method of claim 19, wherein the subject, prior to or at the time of administration of the pharmaceutical composition, exhibits hypercholesterolemia defined as a serum low-density lipoprotein cholesterol (LDL-C) level of greater than about 70 mg/dL.

23. The method of claim 19, wherein the subject, prior to or at the time of administration of the pharmaceutical composition, exhibits hypercholesterolemia defined as a serum low-density lipoprotein cholesterol (LDL-C) level of greater than about 100 mg/dL.

24. The method of claim 19, wherein the subject has heterozygous Familial Hypercholesterolemia (heFH).

25. The method of claim 19, wherein the subject has a form of hypercholesterolemia that is not Familial Hypercholesterolemia (nonFH).

26. The method of claim 19, wherein the background lipid-modifying therapy comprises a therapeutic agent selected from the group consisting of a statin, ezetimibe, a fibrate, niacin, an omega-3 fatty acid, and a bile acid resin.

27. The method of claim 19, wherein the background lipid-modifying therapy comprises a statin selected from the group consisting of cerivastatin, atorvastatin, simvastatin, pitavastatin, rosuvastatin, fluvastatin, lovastatin and pravastatin.

28. The method of claim 19, wherein the pharmaceutical composition is administered for at least 24 weeks.

29. The method of claim 19, wherein the pharmaceutical composition is administered subcutaneously.

30. The method of claim 19, wherein the antibody or antigen binding fragment thereof comprises the heavy and light chain CDRs of a HCVR/LCVR amino acid sequence pair selected from the group consisting of SEQ ID NOs: 1/6 and 11/15.

31. The method of claim 19, wherein the antibody or antigen-binding fragment thereof comprises heavy and light chain CDR amino acid sequences having SEQ ID NOs: 12, 13, 14, 16, 17, and 18.

32. The method of claim 19, wherein the antibody or antigen-binding fragment thereof comprises an HCVR having the amino acid sequence of SEQ ID NO: 11 and an LCVR having the amino acid sequence of SEQ ID NO: 15.

33. The method of claim 19, wherein the antibody or antigen-binding fragment thereof comprises heavy and light chain CDR amino acid sequences having SEQ ID NOs: 2, 3, 4, 7, 8, and 10.

34. The method of claim 19, wherein the antibody or antigen-binding fragment thereof comprises an HCVR having the amino acid sequence of SEQ ID NO: 1 and an LCVR having the amino acid sequence of SEQ ID NO: 6.

35. The method of claim 19, wherein the antibody or antigen-binding fragment thereof binds to the same epitope on PCSK9 as an antibody comprising heavy and light chain CDR amino acid sequences having SEQ ID NOs: 12, 13, 14, 16, 17, and 18; or SEQ ID NOs: 2, 3, 4, 7, 8, and 10.

36. The method of claim 19, wherein the antibody or antigen-binding fragment thereof competes for binding to PCSK9 with an antibody comprising heavy and light chain CDR amino acid sequences having SEQ ID NOs: 12, 13, 14, 16, 17, and 18; or SEQ ID NOs: 2, 3, 4, 7, 8, and 10.

37. (canceled)

38. A method for improving one or more hypercholesterolemia-associated parameters in a subject in need thereof comprising administering to the subject a pharmaceutical composition comprising about 75 mg of an antibody or antigen binding fragment thereof that specifically binds PCSK9 at a frequency of about once every other week (q2w), wherein the subject is treated concurrently with a background lipid-modifying therapy, and wherein the improvement in a hypercholesterolemia-associated parameter is selected from the group consisting of:
a) a decrease from baseline of low density lipoprotein-C (LDL-C);
b) a decrease from baseline of total cholesterol (TC); and
c) a decrease from baseline of lipoprotein (a).

39. The method of claim 38, wherein the improvement in a hypercholesterolemia-associated parameter is a decrease from baseline of LDL-C of at least 50%.

40. The method of claim 38, wherein the improvement in a hypercholesterolemia-associated parameter is a decrease from baseline of LDL-C of at least 60%.

41. The method of claim 38, wherein the improvement in a hypercholesterolemia-associated parameter is a decrease from baseline of TC of at least 30%.

42. The method of claim 38, wherein the improvement in a hypercholesterolemia-associated parameter is a decrease from baseline of TC of at least 35%.

43. The method claim 38, wherein the improvement in a hypercholesterolemia-associated parameter is a decrease from baseline of lipoprotein (a) of at least 30%.

44. The method of claim 38, wherein the improvement in a hypercholesterolemia-associated parameter is a decrease from baseline of lipoprotein (a) of at least 40%.

45. A method for lowering serum low-density lipoprotein cholesterol (LDL-C) in a subject in need thereof, comprising:
a) selecting a patient with hypercholesterolemia who is currently being treated for hypercholesterolemia by administration of a background moderate-dose statin therapy, and who is not adequately controlled by moderate-dose statin therapy,
b) continuing to treat the subject with the moderate-dose statin therapy; and
c) administering to the subject a pharmaceutical composition comprising about 75 mg of an antibody or antigen binding fragment thereof that specifically binds PCSK9 at a frequency of about once every other week (q2w), thereby lowering the serum LDL-C level by at least 50%.

46. The method of claim 45, wherein the subject exhibits a serum LDL-C level of greater than about 70 mg/dL.

47. The method of claim 45, wherein the subject exhibits a serum LDL-C level of greater than about 100 mg/dL.

48. The method of claim 45, wherein the subject's serum LDL-C level is lowered by at least 60%.

49. The method of claim 45, wherein the subject has heterozygous Familial Hypercholesterolemia (heFH).

50. The method of claim 45, wherein the subject has a form of hypercholesterolemia that is not Familial Hypercholesterolemia (nonFH).

51. The method of claim 45, wherein the subject is also being treated with a non-statin background lipid-modifying therapy comprising a therapeutic agent selected from the group consisting of ezetimibe, a fibrate, niacin, an omega-3 fatty acid, and a bile acid resin.

52. The method of claim 45, wherein the statin is selected from the group consisting of cerivastatin, atorvastatin, simvastatin, pitavastatin, rosuvastatin, fluvastatin, lovastatin and pravastatin.

53. The method of claim 45, wherein the pharmaceutical composition is administered for at least 24 weeks.

54. The method of claim 45, wherein the pharmaceutical composition is administered subcutaneously.

55. The method of claim 45, wherein the antibody or antigen binding fragment thereof comprises the heavy and light chain CDRs of a HCVR/LCVR amino acid sequence pair selected from the group consisting of SEQ ID NOs: 1/6 and 11/15.

56. The method of claim 45, wherein the antibody or antigen-binding fragment thereof comprises heavy and light chain CDR amino acid sequences having SEQ ID NOs: 12, 13, 14, 16, 17, and 18.

57. The method of claim 45, wherein the antibody or antigen-binding fragment thereof comprises an HCVR having the amino acid sequence of SEQ ID NO: 11 and an LCVR having the amino acid sequence of SEQ ID NO: 15.

58. The method of claim 45, wherein the antibody or antigen-binding fragment thereof comprises heavy and light chain CDR amino acid sequences having SEQ ID NOs: 2, 3, 4, 7, 8, and 10.

59. The method of claim 45, wherein the antibody or antigen-binding fragment thereof comprises an HCVR having the amino acid sequence of SEQ ID NO: 1 and an LCVR having the amino acid sequence of SEQ ID NO: 6.

60. The method of claim 45, wherein the antibody or antigen-binding fragment thereof binds to the same epitope on PCSK9 as an antibody comprising heavy and light chain CDR amino acid sequences having SEQ ID NOs: 12, 13, 14, 16, 17, and 18; or SEQ ID NOs: 2, 3, 4, 7, 8, and 10.

61. The method of claim 45, wherein the antibody or antigen-binding fragment thereof competes for binding to PCSK9 with an antibody comprising heavy and light chain CDR amino acid sequences having SEQ ID NOs: 12, 13, 14, 16, 17, and 18; or SEQ ID NOs: 2, 3, 4, 7, 8, and 10.


SEQUENCE LISTING (198 sequences)

SEQ ID NO: 1 - protein, 118 aa, Artificial Sequence [sequence omitted]

SEQ ID NO: 2 - protein, 8 aa, Artificial Sequence
  Description of Artificial Sequence Synthetic peptide
GFTFNNYA

SEQ ID NO: 3 - protein, 8 aa, Artificial Sequence
  Description of Artificial Sequence Synthetic peptide
ISGSGGTT

SEQ ID NO: 4 - protein, 11 aa, Artificial Sequence
  Description of Artificial Sequence Synthetic peptide
AKDSNWGNFDL

SEQ ID NO: 5 - protein, 447 aa, Artificial Sequence
  Description of Artificial Sequence Synthetic REGN727 heavy chain polypeptide
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 113 aa, Artificial Sequence [sequence omitted]

SEQ ID NO: 7 - protein, 12 aa, Artificial Sequence
  Description of Artificial Sequence Synthetic peptide
QSVLYRSNNRNF

SEQ ID NO: 8 - protein, 3 aa, Artificial Sequence
  Description of Artificial Sequence Synthetic peptide
WAS

SEQ ID NO: 9 - protein, 220 aa, Artificial Sequence
  Description of Artificial Sequence Synthetic REGN727 light chain polypeptide
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 9 aa, Artificial Sequence
  Description of Artificial Sequence Synthetic peptide
QQYYTTPYT

SEQ ID NO: 11 - protein, 127 aa, Artificial Sequence
  Description of Artificial Sequence Synthetic polypeptide
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 12 - protein, 8 aa, Artificial Sequence
  Description of Artificial Sequence Synthetic peptide
GFTFSSHW

SEQ ID NO: 13 - protein, 8 aa, Artificial Sequence
  Description of Artificial Sequence Synthetic peptide
INQDGSEK

SEQ ID NO: 14 - protein, 20 aa, Artificial Sequence
  Description of Artificial Sequence Synthetic peptide
ARDIVLMVYDMDYYYYGMDV

SEQ ID NO: 15 - protein, 112 aa, Artificial Sequence
  Description of Artificial Sequence Synthetic polypeptide
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK

SEQ ID NO: 16 - protein, 11 aa, Artificial Sequence
  Description of Artificial Sequence Synthetic peptide
QSLLHSNGNNY

SEQ ID NO: 17 - protein, 3 aa, Artificial Sequence
  Description of Artificial Sequence Synthetic peptide
LGS

SEQ ID NO: 18 - protein, 9 aa, Artificial Sequence
  Description of Artificial Sequence Synthetic peptide
MQTLQTPLT

SEQ ID NO: 19 - protein, 8 aa, Artificial Sequence
  Description of Artificial Sequence Synthetic peptide
GFTFSSHW

SEQ ID NO: 20 - protein, 8 aa, Artificial Sequence
  Description of Artificial Sequence Synthetic peptide
INQDGSEK

SEQ ID NO: 21 - protein, 20 aa, Artificial Sequence
  Description of Artificial Sequence Synthetic peptide
ARDIVLMVYDMDYYYYGMDV

SEQ ID NO: 22 - protein, 11 aa, Artificial Sequence
  Description of Artificial Sequence Synthetic peptide
QSLHHSNGNNY

SEQ ID NO: 23 - protein, 3 aa, Artificial Sequence
  Description of Artificial Sequence Synthetic peptide
LGS

SEQ ID NO: 24 - protein, 9 aa, Artificial Sequence
  Description of Artificial Sequence Synthetic peptide
MQTLQTPLT

SEQ ID NO: 25 - protein, 8 aa, Artificial Sequence
  Description of Artificial Sequence Synthetic peptide
GFTFSSHW

SEQ ID NO: 26 - protein, 8 aa, Artificial Sequence
  Description of Artificial Sequence Synthetic peptide
INQDGSEK

SEQ ID NO: 27 - protein, 20 aa, Artificial Sequence
  Description of Artificial Sequence Synthetic peptide
ARDIVLMVYHMDYYYYGMDV

SEQ ID NO: 28 - protein, 11 aa, Artificial Sequence
  Description of Artificial Sequence Synthetic peptide
QSLLHSNGNNY

SEQ ID NO: 29 - protein, 3 aa, Artificial Sequence
  Description of Artificial Sequence Synthetic peptide
LGS

SEQ ID NO: 30 - protein, 9 aa, Artificial Sequence
  Description of Artificial Sequence Synthetic peptide
MQTLQTPLT

SEQ ID NO: 31 - protein, 8 aa, Artificial Sequence
  Description of Artificial Sequence Synthetic peptide
GFTFSSHW

SEQ ID NO: 32 - protein, 8 aa, Artificial Sequence
  Description of Artificial Sequence Synthetic peptide
INQDGSEK

SEQ ID NO: 33 - protein, 20 aa, Artificial Sequence
  Description of Artificial Sequence Synthetic peptide
ARDIVLMVYHMDYYYYGMDV

SEQ ID NO: 34 - protein, 11 aa, Artificial Sequence
  Description of Artificial Sequence Synthetic peptide
QSLHHSNGNNY

SEQ ID NO: 35 - protein, 3 aa, Artificial Sequence
  Description of Artificial Sequence Synthetic peptide
LGS

SEQ ID NO: 36 - protein, 9 aa, Artificial Sequence
  Description of Artificial Sequence Synthetic peptide
MQTLQTPLT

SEQ ID NO: 37 - protein, 131 aa, Artificial Sequence
  Description of Artificial Sequence Synthetic VH; m2CX1D05 polypeptide
QVQLVQSGAEVKKPGSSVKVSCKASGGTFNSHAISWVRQAPGQGLEWMGGINPILGIANYAQKFQGRVTITADESTSTAYMELSSLRSEDTAVYYCARHYEIQIGRYGMNVYYLMYRFASWGQGTLVTVSS

SEQ ID NO: 38 - protein, 10 aa, Artificial Sequence
  Description of Artificial Sequence Synthetic VH CDR1; m2CX1D05 peptide
GGTFNSHAIS

SEQ ID NO: 39 - protein, 20 aa, Artificial Sequence
  Description of Artificial Sequence Synthetic VH CDR2; m2CX1D05 peptide
WMGGINPILGIANYAQKFQG

SEQ ID NO: 40 - protein, 22 aa, Artificial Sequence
  Description of Artificial Sequence Synthetic VH CDR3; m2CX1D05 peptide
HYEIQIGRYGMNVYYLMYRFAS

SEQ ID NO: 41 - protein, 213 aa, Artificial Sequence
  Description of Artificial Sequence Synthetic LC; m2CX1D05 polypeptide
DIQMTQSPSSLSASVGDRVTITCRASQGIRSALNWYQQKPGKAPKLLIYNGSTLQSGVPSRFSGSGSGTDFTLTISSLQPEDFAVYYCQQFDGDPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 42 - protein, 11 aa, Artificial Sequence
  Description of Artificial Sequence Synthetic VL CDR 1; m2CX1D05 peptide
RASQGIRSALN

SEQ ID NO: 43 - protein, 11 aa, Artificial Sequence
  Description of Artificial Sequence Synthetic VL CDR2; m2CX1D05 peptide
LLIYNGSTLQS

SEQ ID NO: 44 - protein, 7 aa, Artificial Sequence
  Description of Artificial Sequence Synthetic VL CDR3; m2CX1D05 peptide
QQFDGDP

SEQ ID NO: 45 - protein, 119 aa, Artificial Sequence
  Description of Artificial Sequence Synthetic VH; 1B20 polypeptide
QVQLVQSGAEVKKPGESLKISCKGSGYSFTNYWISWVRQMPGKGLEWMGIIYPGDSYTNYSPSFQGQVTISADKSISTAYLQWSSLKASDTAMYYCARDYWYKPLFDIWGQGTLVTVSS

SEQ ID NO: 46 - protein, 10 aa, Artificial Sequence
  Description of Artificial Sequence Synthetic VH CDR1; 1B20 peptide
GYSFTNYWIS

SEQ ID NO: 47 - protein, 20 aa, Artificial Sequence
  Description of Artificial Sequence Synthetic VH CDR2; 1B20 peptide
WMGIIYPGDSYTNYSPSFQG

SEQ ID NO: 48 - protein, 10 aa, Artificial Sequence
  Description of Artificial Sequence Synthetic VH CDR3; 1B20 peptide
DYWYKPLFDI

SEQ ID NO: 49 - protein, 220 aa, Artificial Sequence
  Description of Artificial Sequence Synthetic LC; 1B20 polypeptide
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYSSFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 50 - protein, 17 aa, Artificial Sequence
  Description of Artificial Sequence Synthetic VL CDR1; 1B20 peptide
RSSQSVLYSSNNKNYLA

SEQ ID NO: 51 - protein, 11 aa, Artificial Sequence
  Description of Artificial Sequence Synthetic VL CDR2; 1B20 peptide
LLIYWASTRES

SEQ ID NO: 52 - protein, 8 aa, Artificial Sequence
  Description of Artificial Sequence Synthetic VL CDR3; 1B20 peptide
QQYSSFPI

SEQ ID NO: 53 - protein, 120 aa, Artificial Sequence
  Description of Artificial Sequence Synthetic variable heavy antibody region polypeptide
EVQLLESGGGLVQPGGSLRLSCKASGYTFSSYGMYWVRQAPGKGLEWIGWIDPGSGGTKYNEKFKGKATISRDNSKNTLYLQMNSLRAEDTAVYYCARERYGYYFDYWGQGTLVTVSSAS

SEQ ID NO: 54 - protein, 16 aa, Artificial Sequence
  Description of Artificial Sequence Synthetic AX132 heavy chain CDR1 antibody region peptide
KASGYTFSSYGMYWVR

SEQ ID NO: 55 - protein, 23 aa, Artificial Sequence
  Description of Artificial Sequence Synthetic AX132 heavy chain CDR2 antibody region peptide
WIGWIDPGSGGTKYNEKFKGKAT

SEQ ID NO: 56 - protein, 15 aa, Artificial Sequence
  Description of Artificial Sequence Synthetic AX132 heavy chain CDR3 antibody region peptide
CARERYGYYFDYWGQ

SEQ ID NO: 57 - protein, 108 aa, Artificial Sequence
  Description of Artificial Sequence Synthetic variable light antibody region polypeptide
EIVLTQSPATLSLSPGERATITCRASQYVGSYLNWYQQKPGQAPRLLIYDASNRATGIPARFSGSGSGTDFTLTISSLEPEDFAVYYCQVWDSSPPVVFGGGTKVEIK

SEQ ID NO: 58 - protein, 17 aa, Artificial Sequence
  Description of Artificial Sequence Synthetic AX213 and AX132 light chain CDR1 antibody region peptide
ITCRASQYVGSYLNWYQ

SEQ ID NO: 59 - protein, 13 aa, Artificial Sequence
  Description of Artificial Sequence Synthetic AX213 and AX132 light chain CDR2 antibody region peptide
LIYDASNRATGIP

SEQ ID NO: 60 - protein, 16 aa, Artificial Sequence
  Description of Artificial Sequence Synthetic AX132 & AX213 light chain CDR3 antibody region peptide
YYCQVWDSSPPVVFGG

SEQ ID NO: 61 - protein, 120 aa, Artificial Sequence
  Description of Artificial Sequence Synthetic variable heavy antibody region polypeptide
EVQLLESGGGLVQPGGSLRLSCKASGYTFSRYGINWVRQAPGKGLEWIGRIDPGNGGTRYNEKFKGKATISRDNSKNTLYLQMNSLRAEDTAVYYCARANDGYSFDYWGQGTLVTVSSAS

SEQ ID NO: 62 - protein, 16 aa, Artificial Sequence
  Description of Artificial Sequence Synthetic AX213 heavy chain CDR1 antibody region peptide
KASGYTFSRYGINWVR

SEQ ID NO: 63 - protein, 23 aa, Artificial Sequence
  Description of Artificial Sequence Synthetic AX213 heavy chain CDR2 antibody region peptide
WIGRIDPGNGGTRYNEKFKGKAT

SEQ ID NO: 64 - protein, 15 aa, Artificial Sequence
  Description of Artificial Sequence Synthetic AX213 heavy chain CDR3 antibody region peptide
CARANDGYSFDYWGQ

SEQ ID NO: 65 - protein, 108 aa, Artificial Sequence; identical to SEQ ID NO: 57
  Description of Artificial Sequence Synthetic variable light antibody region polypeptide

SEQ ID NO: 66 - protein, 17 aa, Artificial Sequence
  Description of Artificial Sequence Synthetic AX213 and AX132 light chain CDR1 antibody region peptide
ITCRASQYVGSYLNWYQ

SEQ ID NO: 67 - protein, 13 aa, Artificial Sequence
  Description of Artificial Sequence Synthetic AX213 and AX132 light chain CDR2 antibody region peptide
LIYDASNRATGIP

SEQ ID NO: 68 - protein, 16 aa, Artificial Sequence
  Description of Artificial Sequence Synthetic AX132 & AX213 light chain CDR3 antibody region peptide
YYCQVWDSSPPVVFGG

SEQ ID NO: 69 - protein, 119 aa, Artificial Sequence
  Description of Artificial Sequence Synthetic AX1 VH antibody sequence polypeptide
EVQLLESGGGLVQPGGSLRLSCKASGFTFTSYYMHWVRQAPGKGLEWIGRINPDSGSTKYNEKFKGRATISRDNSKNTLYLQMNSLRAEDTAVYYCARGGRLSWDFDVWGQGTLVTVSS

SEQ ID NO: 70 - protein, 16 aa, Artificial Sequence
  Description of Artificial Sequence Synthetic AX1 VH CDR1 antibody sequence peptide
KASGFTFTSYYMHWVR

SEQ ID NO: 71 - protein, 23 aa, Artificial Sequence
  Description of Artificial Sequence Synthetic AX1 VH CDR2 antibody sequence peptide
WIGRINPDSGSTKYNEKFKGRAT

SEQ ID NO: 72 - protein, 16 aa, Artificial Sequence
  Description of Artificial Sequence Synthetic AX1 VH CDR3 antibody sequence peptide
CARGGRLSWDFDVWGQ

SEQ ID NO: 73 - protein, 109 aa, Artificial Sequence
  Description of Artificial Sequence Synthetic AX1 VL antibody sequence polypeptide
DIQMTQSPSSLSASVGDRVTITCRASQDISRYLAWYQQKPGKAPKLLIYAASSLQSGVPSRFSGSGSGTDFTLTISSLQPEDFATYYCAAYDYSLGGYVFGDGTKVEIK

SEQ ID NO: 74 - protein, 11 aa, Artificial Sequence
  Description of Artificial Sequence Synthetic AX1 VL CDR1 antibody sequence peptide
RASQDISRYLA

SEQ ID NO: 75 - protein, 7 aa, Artificial Sequence
  Description of Artificial Sequence Synthetic AX1 AX9 AX189 VL CDR2 antibody sequence peptide
AASSLQS

SEQ ID NO: 76 - protein, 11 aa, Artificial Sequence
  Description of Artificial Sequence Synthetic AX1 VL CDR3 antibody sequence peptide
AAYDYSLGGYV

SEQ ID NO: 77 - protein, 121 aa, Artificial Sequence
  Description of Artificial Sequence Synthetic AX9 AX189 VH antibody sequence polypeptide
EVQLLESGGGLVQPGGSLRLSCKASGYTFSSYWMHWVRQAPGKGLEWIGRIDPYNGGTKYNEKFKGKATISRDNSKNTLYLQMNSLRAEDTAVYYCARYGYYLGSYAMDYWGQGTLVTVSS

SEQ ID NO: 78 - protein, 16 aa, Artificial Sequence
  Description of Artificial Sequence Synthetic AX9 AX189 VH CDR1 antibody sequence peptide
KASGYTFSSYWMHWVR

SEQ ID NO: 79 - protein, 23 aa, Artificial Sequence
  Description of Artificial Sequence Synthetic AX9 AX189 VH CDR2 antibody sequence peptide
WIGRIDPYNGGTKYNEKFKGKAT

SEQ ID NO: 80 - protein, 18 aa, Artificial Sequence
  Description of Artificial Sequence Synthetic AX9 AX189 VH CDR3 antibody sequence peptide
CARYGYYLGSYAMDYWGQ

SEQ ID NO: 81 - protein, 109 aa, Artificial Sequence
  Description of Artificial Sequence Synthetic AX189 VL antibody sequence polypeptide
DIQMTQSPSSLSASVGDRVTITCRASQDVSRYLTWYQQKPGKAPKLLIYAASSLQSGVPSRFSGSGSGTDFTLTISSLQPEDFATYYCQAYDYSLSGYVFGGGTKVEIK

SEQ ID NO: 82 - protein, 11 aa, Artificial Sequence
  Description of Artificial Sequence Synthetic AX189 VL CDR1 antibody sequence peptide
RASQDVSRYLT

SEQ ID NO: 83 - protein, 7 aa, Artificial Sequence
  Description of Artificial Sequence Synthetic AX1 AX9 AX189 VL CDR2 antibody sequence peptide
AASSLQS

SEQ ID NO: 84 - protein, 11 aa, Artificial Sequence
  Description of Artificial Sequence Synthetic AX189 VL CDR3 antibody sequence peptide
QAYDYSLSGYV

SEQ ID NO: 85 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 86 - protein, 5 aa, Homo sapiens
SYGIS

SEQ ID NO: 87 - protein, 17 aa, Homo sapiens
WVSFYNGNTNYAQKLQG

SEQ ID NO: 88 - protein, 6 aa, Homo sapiens
GYGMDV

SEQ ID NO: 89 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 90 - protein, 14 aa, Homo sapiens
TGTSSDVGGYNSVS

SEQ ID NO: 91 - protein, 7 aa, Homo sapiens
EVSNRPS

SEQ ID NO: 92 - protein, 9 aa, Homo sapiens
NSYTSTSMV

SEQ ID NO: 93 - protein, 123 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 94 - protein, 10 aa, Homo sapiens
GFTFSSYSMN

SEQ ID NO: 95 - protein, 17 aa, Homo sapiens
SISSSSSYISYADSVKG

SEQ ID NO: 96 - protein, 14 aa, Homo sapiens
DYDFWSAYYDAFDV

SEQ ID NO: 97 - protein, 111 aa, Homo sapiens
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 98 - protein, 14 aa, Homo sapiens
TGSSSNIGAGYDVH

SEQ ID NO: 99 - protein, 7 aa, Homo sapiens
GNSNRPS

SEQ ID NO: 100 - protein, 11 aa, Homo sapiens
QSYDSSLSGSV

SEQ ID NO: 101 - protein, 114 aa, Homo sapiens
QVQLVESGGGVAQPGRSLRLSCAASGFTFSSYGMHWVRQAPGKGLEWVAVIYYDGINKHYADSVKGRFTISRDNSKNTLYLQMNSLRAEDTAVYYCARDRGLDWGQGTLVTVSS

SEQ ID NO: 102 - protein, 10 aa, Homo sapiens
GFTFSSYGMH

SEQ ID NO: 103 - protein, 17 aa, Homo sapiens
VIYYDGINKHYADSVKG

SEQ ID NO: 104 - protein, 5 aa, Homo sapiens
DRGLD

SEQ ID NO: 105 - protein, 113 aa, Homo sapiens
DIVMTQSPDSLAVSLGERATINCKSSQSVLYSSNSKNYLVWYQQKPGQPPKLLIYWASTRESGVPDRFSGSGSGTDFTLTISSLQAEDVAVYYCQQYYSTPWTFGQGTKVEIK

SEQ ID NO: 106 - protein, 17 aa, Homo sapiens
KSSQSVLYSSNSKNYLV

SEQ ID NO: 107 - protein, 7 aa, Homo sapiens
WASTRES

SEQ ID NO: 108 - protein, 9 aa, Homo sapiens
QQYYSTPWT

SEQ ID NO: 109 - protein, 118 aa, Artificial Sequence
  Description of Artificial Sequence Synthetic anti-PCSK9 monoclonal antibody pJG04 (clones LGT-209 and LGT-210) Vh heavy chain variable region (FR1-FR4) polypeptide
QVQLVQSGAEVKKPGASVKVSCKASGYTFSTMYMSWVRQAPGQGLEWMGRIDPANEHTNYAQKFQGRVTMTRDTSISTAYMELSRLTSDDTAVYYCARSYYYYNMDYWGQGTLVTVSS

SEQ ID NO: 110 - protein, 5 aa, Artificial Sequence
  Description of Artificial Sequence Synthetic anti-PCSK9 monoclonal antibody clones LGT-209, LGT-210 and LGT-211 heavy chain CDR1 peptide
TMYMS

SEQ ID NO: 111 - protein, 17 aa, Artificial Sequence
  Description of Artificial Sequence Synthetic anti-PCSK9 monoclonal antibody clones LGT-209, LGT-210 and LGT-211 heavy chain CDR2 peptide
RIDPANEHTNYAQKFQG

SEQ ID NO: 112 - protein, 9 aa, Artificial Sequence
  Description of Artificial Sequence Synthetic anti-PCSK9 monoclonal antibody pJG04(clones LGT-209 and LGT-210) Vh heavy chain complementarity determining region 3 (CDR3) peptide
SYYYYNMDY

SEQ ID NO: 113 - protein, 106 aa, Artificial Sequence
  Description of Artificial Sequence Synthetic anti-PCSK9 monoclonal antibody pJG10(clones LGT-209 and LGT-211) Vk light chain variable region (FR1-FR4) polypeptide
QIVLTQSPATLSVSPGERATLSCRASQSVSYMHWYQQKPGQAPRLLIYGVFRRATGIPDRFSGSGSGTDFTLTIGRLEPEDFAVYYCLQWSSDPPTFGQGTKLEIK

SEQ ID NO: 114 - protein, 10 aa, Artificial Sequence
  Description of Artificial Sequence Synthetic anti-PCSK9 monoclonal antibody clones LGT-209, LGT-210 and LGT-211 light chain CDR1 peptide
RASQSVSYMH

SEQ ID NO: 115 - protein, 7 aa, Artificial Sequence
  Description of Artificial Sequence Synthetic anti-PCSK9 monoclonal antibody clones LGT-209, LGT-210 and LGT-211 light chain CDR1 peptide
GVFRRAT

SEQ ID NO: 116 - protein, 9 aa, Artificial Sequence
  Description of Artificial Sequence Synthetic mouse anti-PCSK9 monoclonal antibody LFU720 and anti-PCSK9 monoclonal antibody clones LGT-209, LGT-210 and LGT-211 light chain CDR3 peptide
LQWSSDPPT

SEQ ID NO: 117 - protein, 118 aa, Homo sapiens
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 118 - protein, 7 aa, Artificial Sequence
  Description of Artificial Sequence Synthetic peptide
GYTFTSY

SEQ ID NO: 119 - protein, 6 aa, Artificial Sequence
  Description of Artificial Sequence Synthetic peptide
SPFGGR

SEQ ID NO: 120 - protein, 9 aa, Artificial Sequence
  Description of Artificial Sequence Synthetic variable heavy chain CDR peptide
ERPLYASDL

SEQ ID NO: 121 - protein, 107 aa, Homo sapiens
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 122 - protein, 11 aa, Artificial Sequence
  Description of Artificial Sequence Synthetic variable light chain CDR peptide
RASQGISSALA

SEQ ID NO: 123 - protein, 7 aa, Artificial Sequence
  Description of Artificial Sequence Synthetic variable light chain CDR peptide
SASYRYT

SEQ ID NO: 124 - protein, 9 aa, Artificial Sequence
  Description of Artificial Sequence Synthetic variable light chain CDR peptide
QQRYSLWRT

SEQ ID NO: 125 - protein, 118 aa, Homo sapiens
QVQLVQSGAEVKKPGASVKVSCKASGYTFTSYYMHWVRQAPGQGLEWMGEIHPSGGRTNYNEKFKSRVTMTRDTSTSTVYMELSSLRSEDTAVYYCARERPLYAMDYWGQGTTVTVSS

SEQ ID NO: 126 - protein, 10 aa, Homo sapiens
GYTFTSYYMH

SEQ ID NO: 127 - protein, 17 aa, Homo sapiens
EIHPSGGRTNYNEKFKS

SEQ ID NO: 128 - protein, 9 aa, Homo sapiens
ERPLYAMDY

SEQ ID NO: 129 - protein, 107 aa, Homo sapiens [sequence omitted]

SEQ ID NO: 130 - protein, 11 aa, Homo sapiens
KASQDVHTAVA

SEQ ID NO: 131 - protein, 7 aa, Homo sapiens
HASYRYT

SEQ ID NO: 132 - protein, 9 aa, Homo sapiens
QQRYSLWRT

SEQ ID NO: 133 - protein, 118 aa, Homo sapiens [sequence omitted]

SEQ ID NO: 134 - protein, 10 aa, Homo sapiens
GYTFTSYYMH

SEQ ID NO: 135 - protein, 17 aa, Homo sapiens
EIHPSGGRTNYNEKFKS

SEQ ID NO: 136 - protein, 9 aa, Homo sapiens
ERPLYASDL

SEQ ID NO: 137 - protein, 107 aa, Homo sapiens [sequence omitted]

SEQ ID NO: 138 - protein, 11 aa, Homo sapiens
KASQDVHTAVA

SEQ ID NO: 139 - protein, 7 aa, Homo sapiens
HASYRYT

SEQ ID NO: 140 - protein, 9 aa, Homo sapiens
QQRYSLWRT

SEQ ID NO: 141 - protein, 118 aa, Mus musculus
QVQLQQPGAELVKPGASVKLSCKASGYTFTSYWMHWVKQRPGQGLEWIGEINPSNGRTNYNEKFKSKATLTVDKSSSTAYMQLSSLTSEDSAVYYCARERPLYAMDYWGQGTSVTVSS

SEQ ID NO: 142 - protein, 7 aa, Mus musculus
GYTFTSY

SEQ ID NO: 143 - protein, 6 aa, Mus musculus
NPSNGR

SEQ ID NO: 144 - protein, 9 aa, Mus musculus
ERPLYAMDY

SEQ ID NO: 145 - protein, 108 aa, Mus musculus
DIVMTQSHKFMSTSVGDRVSITCKASQDVSTAVAWYQQKPGQSPKLLIYSASYRYTGVPDRFTGSGSGTDFTFTISSVQAEDLAVYYCQQRYSTPRTFGGGTKLEIKR

SEQ ID NO: 146 - protein, 11 aa, Mus musculus
KASQDVSTAVA

SEQ ID NO: 147 - protein, 7 aa, Artificial Sequence
  Description of Artificial Sequence Synthetic variable light chain CDR peptide
SASYRYT

SEQ ID NO: 148 - protein, 9 aa, Mus musculus
QQRYSTPRT

SEQ ID NO: 149 - protein, 115 aa, Mus musculus
EVQLQQSGPELVKPGASVKISCKASGYTFTDYYMNWVKQSHGKSLEWIGDINPNNGGTTYNQKFKGKATLTVDKSYSTAYMELRSLTSEDSAVYYCARWLLFAYWGQGTLVTVSA

SEQ ID NO: 150 - protein, 7 aa, Mus musculus
GYTFTDY

SEQ ID NO: 151 - protein, 6 aa, Mus musculus
NPNNGG

SEQ ID NO: 152 - protein, 6 aa, Mus musculus
WLLFAY

SEQ ID NO: 153 - protein, 108 aa, Mus musculus
DIVMTQSQKFMSTSVGDRVSVTCKASQNVGTNVAWYQQKPGQSPKALIYSASYRYSGVPDRFTGSGSGTDFTLTISNVLSEDLAEYFCQQFYSYPYTFGGGTKLEIKR

SEQ ID NO: 154 - protein, 11 aa, Mus musculus
KASQNVGTNVA

SEQ ID NO: 155 - protein, 7 aa, Mus musculus
SASYRYS

SEQ ID NO: 156 - protein, 9 aa, Mus musculus
QQFYSYPYT

SEQ ID NO: 157 - protein, 123 aa, Mus musculus
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAGGGIYYRYDRNYFDYWGQGTTLTVSS

SEQ ID NO: 158 - protein, 7 aa, Mus musculus
GYTFTDY

SEQ ID NO: 159 - protein, 6 aa, Mus musculus
NPNNGG

SEQ ID NO: 160 - protein, 14 aa, Mus musculus
GGIYYRYDRNYFDY

SEQ ID NO: 161 - protein, 107 aa, Mus musculus
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPFTFGSGTKLEIK

SEQ ID NO: 162 - protein, 11 aa, Mus musculus
SASQGISNYLN

SEQ ID NO: 163 - protein, 7 aa, Mus musculus
YTSSLHS

SEQ ID NO: 164 - protein, 9 aa, Artificial Sequence
  Description of Artificial Sequence Synthetic peptide
QQYSKLPFT

SEQ ID NO: 165 - protein, 117 aa, Mus musculus
EVKLVESEGGLVQPGSSMKLSCTASGFTFSDYYMAWVRQVPEKGLEWVANINYDGSNTSYLDSLKSRFIISRDNAKNILYLQMSSLKSEDTATYYCAREKFAAMDYWGQGTSVTVSS

SEQ ID NO: 166 - protein, 7 aa, Mus musculus
GFTFSDY

SEQ ID NO: 167 - protein, 6 aa, Mus musculus
NYDGSN

SEQ ID NO: 168 - protein, 8 aa, Mus musculus
EKFAAMDY

SEQ ID NO: 169 - protein, 108 aa, Mus musculus
DIVMTQSHKFMSTSFGDRVSITCKASQDVSNALAWYQQKPGHSPKLLIFSASYRYTGVPDRFTGSGSGTDFTFTISSVQAEDLAVYYCQQHYSTPWTFGGGTKLEIKR

SEQ ID NO: 170 - protein, 11 aa, Mus musculus
KASQDVSNALA

SEQ ID NO: 171 - protein, 7 aa, Artificial Sequence
  Description of Artificial Sequence Synthetic variable light chain CDR peptide
SASYRYT

SEQ ID NO: 172 - protein, 9 aa, Mus musculus
QQHYSTPWT

SEQ ID NO: 173 - protein, 121 aa, Artificial Sequence
  Description of Artificial Sequence Synthetic polypeptide
EVQLVESGGGLVQPGGSLRLSCAASGFTFTRHTIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 174 - protein, 10 aa, Artificial Sequence
  Description of Artificial Sequence Synthetic peptide
GFTFTRHTIH

SEQ ID NO: 175 - protein, 17 aa, Artificial Sequence
  Description of Artificial Sequence Synthetic peptide
RISPANGNTNYADSVKG

SEQ ID NO: 176 - protein, 12 aa, Artificial Sequence
  Description of Artificial Sequence Synthetic peptide
WIGSRELYIMDY

SEQ ID NO: 177 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYRIQPTFGQGTKVEIKR

SEQ ID NO: 178 - protein, 11 aa, Artificial Sequence
  Description of Artificial Sequence Synthetic peptide
RASQDVSTAVA

SEQ ID NO: 179 - protein, 7 aa, Artificial Sequence
  Description of Artificial Sequence Synthetic peptide
SASFLYS

SEQ ID NO: 180 - protein, 9 aa, Artificial Sequence
  Description of Artificial Sequence Synthetic peptide
QQSYRIQPT

SEQ ID NO: 181 - protein, 121 aa, Artificial Sequence
  Description of Artificial Sequence Synthetic polypeptide
EVQLVESGGGLVQPGGSLRLSCAASGFTFSSTAIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 182 - protein, 10 aa, Artificial Sequence
  Description of Artificial Sequence Synthetic peptide
GFTFSSTAIH

SEQ ID NO: 183 - protein, 17 aa, Artificial Sequence
  Description of Artificial Sequence Synthetic peptide
RISPANGNTNYADSVKG

SEQ ID NO: 184 - protein, 12 aa, Artificial Sequence
  Description of Artificial Sequence Synthetic peptide
WIGSRELYIMDY

SEQ ID NO: 185 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYPALHTFGQGTKVEIKR

SEQ ID NO: 186 - protein, 11 aa, Artificial Sequence
  Description of Artificial Sequence Synthetic peptide
RASQDVSTAVA

SEQ ID NO: 187 - protein, 7 aa, Artificial Sequence
  Description of Artificial Sequence Synthetic peptide
SASFLYS

SEQ ID NO: 188 - protein, 9 aa, Artificial Sequence
  Description of Artificial Sequence Synthetic peptide
QQSYPALHT

SEQ ID NO: 189 - protein, 125 aa, Artificial Sequence
  Description of Artificial Sequence Synthetic polypeptide
EVQLVESGGGLVKPGGSLRLSCAASGFPFSKLGMVWVRQAPGKGLEWVSTISSGGGYTYYPDSVKGRFTISRDNAKNSLYLQMNSLRAEDTAVYYCAREGISFQGGTYTYVMDYWGQGTLVTVSS

SEQ ID NO: 190 - protein, 10 aa, Artificial Sequence
  Description of Artificial Sequence Synthetic peptide
GFPFSKLGMV

SEQ ID NO: 191 - protein, 17 aa, Artificial Sequence
  Description of Artificial Sequence Synthetic peptide
TISSGGGYTYYPDSVKG

SEQ ID NO: 192 - protein, 16 aa, Artificial Sequence
  Description of Artificial Sequence Synthetic peptide
EGISFQGGTYTYVMDY

SEQ ID NO: 193 - protein, 112 aa, Artificial Sequence
  Description of Artificial Sequence Synthetic polypeptide
DIVMTQSPLSLPVTPGEPASISCRSSKSLLHRNGITYSYWYLQKPGQSPQLLIYQLSNLASGVPDRFSGSGSGTDFTLKISRVEAEDVGVYYCYQNLELPLTFGQGTKVEIK

SEQ ID NO: 194 - protein, 16 aa, Artificial Sequence
  Description of Artificial Sequence Synthetic peptide
RSSKSLLHRNGITYSY

SEQ ID NO: 195 - protein, 7 aa, Artificial Sequence
  Description of Artificial Sequence Synthetic peptide
QLSNLAS

SEQ ID NO: 196 - protein, 9 aa, Artificial Sequence
  Description of Artificial Sequence Synthetic peptide
YQNLELPLT

SEQ ID NO: 197 - DNA, 2076 bp, Homo sapiens
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccag

SEQ ID NO: 198 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ